(19) United States
(12) Patent Application Publication
(10) Pub. No.: US 2025/0381248 A1
(43) Pub. Date: Dec. 18, 2025

(54) METHODS OF TREATING A TUMOR

(71) Applicant: LONZA SALES AG, Basel (CH)

(72) Inventor: Aaron NOYES, Cambridge, MA (US)

(21) Appl. No.: 18/877,405

(22) PCT Filed: Jun. 28, 2023
(86) PCT No.: PCT/US2023/069250

§ 371 Date: Dec. 20, 2024

Related U.S. Application Data
(60) Provisional application No. 63/357,552, filed on Jun. 30, 2022.

(51) Int. Cl. A61K 38/20; A61K 47/64; A61P 35/00; C07K 16/28

(52) U.S. Cl. CPC A61K 38/208; A61K 47/642; A61P 35/00; C07K 16/2818

(57) ABSTRACT

Provided herein are methods of preventing or treating a tumor in a subject in need thereof comprising administering to the subject a dose of an IL-12 polypeptide, wherein the amount of the IL-12 polypeptide in the dose is about 0.1 μg to about 30 μg.

### BACKGROUND OF THE DISCLOSURE

Interleukin-12 (IL-12) is a promising anticancer agent that has been hindered by unwanted systemic exposure and unpredictable pharmacology. Surface display of functional IL-12 on engineered extracellular vesicles (EVs) provides a promising means of more targeted deliver of IL-12. However, there remains a need in the art for improved methods of delivering IL-12.

### SUMMARY OF THE DISCLOSURE

Some aspects of the present disclosure are directed to a method of preventing or treating a tumor in a subject in need thereof comprising administering to the subject a dose of an IL-12 polypeptide, wherein the amount of the IL-12 polypeptide in the dose is about 0.1 μg to about 30 μg.

In some aspects, the IL-12 polypeptide is delivered by an extracellular vesicle. In some aspects, the IL-12 polypeptide is associated with an extracellular vesicle.

In some aspects, the dose comprises at least about 0.1 μg, at least about 0.2 μg, at least about 0.3 μg, at least about 0.4 μg, at least about 0.5 μg, at least about 0.6 μg, at least about 0.7 μg, at least about 0.8 μg, at least about 0.9 μg, at least about 1.0 μg, at least about 1.1 μg, at least about 1.2 μg, at least about 1.3 μg, at least about 1.4 μg, at least about 1.5 μg, at least about 1.6 μg, at least about 1.7 μg, at least about 1.8 μg, at least about 1.9 μg, at least about 2.0 μg, at least about 2.1 μg, at least about 2.2 μg, at least about 2.3 μg, at least about 2.4 μg, at least about 2.5 μg, at least about 2.6 μg, at least about 2.7 μg, at least about 2.8 μg, at least about 2.9 μg, at least about 3.0 μg, at least about 3.1 μg, at least about 3.2 μg, at least about 3.3 μg, at least about 3.4 μg, at least about 3.5 μg, at least about 3.6 μg, at least about 3.7 μg, at least about 3.8 μg, at least about 3.9 μg, at least about 4.0 μg, at least about 4.1 μg, at least about 4.2 μg, at least about 4.3 μg, at least about 4.4 μg, at least about 4.5 μg, at least about 4.6 μg, at least about 4.7 μg, at least about 4.8 μg, at least about 4.9 μg, at least about 5.0 μg, at least about 5.1 μg, at least about 5.2 μg, at least about 5.3 μg, at least about 5.4 μg, at least about 5.5 μg, at least about 5.6 μg, at least about 5.7 μg, at least about 5.8 μg, at least about 5.9 μg, at least about 6.0 μg, at least about 6.5 μg, at least about 7.0 μg, at least about 7.5 μg, at least about 8.0 μg, at least about 8.5 μg, at least about 9.0 μg, at least about 9.5 μg, at least about 10.0 μg, at least about 10.5 μg, at least about 11.0 μg, at least about 11.5 μg, at least about 12.0 μg, at least about 12.5 μg, at least about 13.0 μg, at least about 14 μg, at least about 15 μg, at least about 16 μg, at least about 17 μg, at least about 18 μg, at least about 19 μg, at least about 20 μg, at least about 25 μg, or at least about 30 μg of the IL-12 polypeptide. In some aspects, the dose comprises at least about 0.3 μg of the IL-12 polypeptide. In some aspects, the dose comprises at least about 1.0 μg of the IL-12 polypeptide. In some aspects, the dose comprises at least about 3.0 μg of the IL-12 polypeptide. In some aspects, the dose comprises at least about 6.0 μg of the IL-12 polypeptide. In some aspects, the dose comprises at least about 12.0 μg of the IL-12 polypeptide.

In some aspects, the dose of the IL-12 polypeptide is administered at least two times, at least three times, at least four times, at least five times, or at least six times. In some aspects, the dose of the IL-12 polypeptide is administered once about every week, once about every two weeks, once about every three weeks, once about every four weeks, once about every six weeks, or once about every eight weeks.

In some aspects, the IL-12 polypeptide comprises a single chain polypeptide having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 13.

In some aspects, the extracellular vesicle comprises a scaffold moiety. In some aspects, the scaffold moiety comprises a Prostaglandin F2 receptor negative regulator (PTGFRN) protein or a portion thereof. In some aspects, the PTGFRN protein comprises SEQ ID NO: 33. In some aspects, the PTGFRN protein comprises at least about 70%, at least about 85%, at least about 90%, at least about 95%, at least about 96%, at least about 97%, at least about 98%, at least about 99%, or about 100% identical to SEQ ID NO: 1. In some aspects, the PTGFRN protein comprises the amino acid sequence as set forth in SEQ ID NO: 1. In some aspects, the IL-12 polypeptide is linked to the scaffold moiety. In some aspects, the IL-12 polypeptide is linked to the scaffold moiety by a peptide bond.

In some aspects, the IL-12 is associated with the exterior surface of the extracellular vesicle. In some aspects, the IL-12 polypeptide is linked to the N-terminus of the scaffold moiety. In some aspects, the IL-12 polypeptide is linked to the N-terminus of the PTGFRN protein of the portion thereof. In some aspects, the IL-12 is within the lumen of the extracellular vesicle. In some aspects, the IL-12 polypeptide is linked to the C-terminus of the scaffold moiety. In some aspects, the IL-12 polypeptide is linked to the C-terminus of the PTGFRN protein of the portion thereof.

In some aspects, the amount of IL-12 polypeptide is measured by fluorescence assay, IL-12 AlphaLISA, or a combination thereof.

In some aspects, the tumor is a primary tumor, a secondary tumor, or both a primary tumor and a secondary tumor. In some aspects, the administering reduces the volume of the tumor. In some aspects, the administering reduces the volume of the tumor by at least two fold, at least three fold, at least four fold, at least five fold, at least six fold, at least seven fold, at least nine fold, or at least ten fold compared to the tumor volume after administering the IL-12 polypeptide in the absence of an extracellular vesicle. In some aspects, the administering reduces the volume of the primary tumor. In some aspects, the administering is capable of reducing the volume of the primary tumor by at least about 1.5 fold, at least about 2 fold, at least about 3 fold, at least about 4 fold, or at least about 5 fold compared to the monotherapy after day 14 of the administering. In some aspects, the administering reduces the growth of the tumor.

In some aspects, the administering reduces the growth of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 polypeptide (“monotherapy”).

In some aspects, the method further comprises administering an additional anti-cancer agent. In some aspects, the additional anti-cancer agent comprises a checkpoint inhibitor. In some aspects, the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof. In some aspects, the checkpoint inhibitor is an anti-PD-1 antibody.

In some aspects, the extracellular vesicle is an exosome, a nanovesicle, an apoptotic body, a microvesicle, a lysosome, an endosome, a liposome, a lipid nanoparticle, a micelle, a multilamellar structure, a revesiculated vesicle, or an extruded cell. In some aspects, the EV is an exosome. In some aspects, the extracellular vesicle is produced by a cell that overexpresses a PTGFRN protein.

In some aspects, the extracellular vesicle further comprises a ligand, a cytokine, or an antibody. In some aspects, the antibody comprises an antagonistic antibody and/or an agonistic antibody.

### BRIEF DESCRIPTION OF THE DRAWINGS

FIG. 1 is a drawing of an engineered exosome having surface displayed IL-12 (“exoIL-12”).

FIGS. 2A-2D are scatter plots showing tumor retention (FIG. 2A), IFN-gamma AUC (FIG. 2B), tumor growth (FIG. 2C), and tumor growth following rechallenge in untreated mice and mice treated with free recombinant IL-12 or exoIL-12, as indicated. FIG. 2E is a box-plot showing the percent of antigen-specific CD8+ T cells in control and exoIL-12-treated mice.

FIGS. 3A-3C are graphical representations the pharmacokinetics, as measured by exoIL-12 measured in the skin and plasma (FIG. 3A), and tissue pharmacodynamics, as measured by the fold change over vehicle of IP-10 in the skin (FIG. 3B) and plasma (FIG. 3C) over time, following administration of 0.3 μg, 1.0 μg, or 3.0 μg of the IL-12 polypeptide.

FIGS. 4A-4B are schematic representations of a clinical study assessing the safety and efficacy of exoIL-12 treatment in healthy volunteers (FIG. 4A) and cancer patients (FIG. 4B).

### DETAILED DESCRIPTION OF THE DISCLOSURE

Before the present invention is described in greater detail, it is to be understood that this invention is not limited to particular aspects described, as such can, of course, vary. It is also to be understood that the terminology used herein is for the purpose of describing particular aspects only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

### I. Definitions

It is noted that, as used herein and in the appended claims, the singular forms “a,” “an,” and “the” include plural referents unless the context clearly dictates otherwise. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The term “about” or “approximately” is used herein to mean approximately roughly, around, or in the region of. When the term “about” is used in conjunction with a numerical range, it modifies that range by extending the boundaries above and below the numerical values set forth. The term used herein means within 5% of the referenced amount, e.g., about 50% is understood to encompass a range of values from 47.5% to 52.5%.

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. Generally extracellular vesicles range in diameter from 20 nm to 1000 nm, and can comprise various macromolecular payload either within the internal space (i.e., lumen), displayed on the external surface of the extracellular vesicle, and/or spanning the membrane. Said payload can comprise nucleic acids, proteins, carbohydrates, lipids, small molecules, and/or combinations thereof. In some aspects, an extracellular vesicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, extracellular vesicles are produced by cells that express one or more transgene products.

As used herein the term “exosome” refers to a cell-derived small (between 20-300 nm in diameter, e.g., 40-200 nm in diameter) vesicle comprising a membrane that encloses an internal space (i.e., lumen), and which is generated from said cell by direct plasma membrane budding or by fusion of the late endosome with the plasma membrane. In some aspects, the EVs, e.g., exosomes, are about 20 nm to about 300 nm. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, an exosome comprises a scaffold moiety. The exosome can be derived from a producer cell, and isolated from the producer cell based on its size, density, biochemical parameters, or a combination thereof. In some aspects, the exosomes of the present disclosure are produced by cells that express one or more transgene products.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, a nanovesicle comprises a scaffold moiety. The nanovesicle, once it is derived from a producer cell according to said manipulation, may be isolated from the producer cell based on its size, density, biochemical parameters, or a combination thereof.

The term “modified,” when used in the context of exosomes described herein, refers to an alteration or engineering of an EV, such that the modified EV is different from a naturally-occurring EV. In some aspects, a modified EV described herein comprises a membrane that differs in composition of a protein, a lipid, a small molecular, a carbohydrate, etc. compared to the membrane of a naturally-occurring EV (e.g., membrane comprises higher density or number of natural EV proteins and/or membrane comprises proteins that are not naturally found in EVs. In certain aspects, such modifications to the membrane changes the exterior surface of the EV. In certain aspects, such modifications to the membrane changes the lumen of the EV.

As used herein, the term “scaffold moiety” refers to a molecule that can be used to anchor an IL-12 moiety and/or any other compound of interest (e.g., payload) to the EV either on the luminal surface or on the exterior surface of the EV. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV. In some aspects, a scaffold moiety comprises a lipid, carbohydrate, or protein that does not naturally exist in the exosome. In certain aspects, a scaffold moiety is Scaffold X. In some aspects, a scaffold moiety is Scaffold Y. In further aspects, a scaffold moiety comprises both Scaffold X and Scaffold Y. In certain aspects, a scaffold moiety comprises Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta, tetraspanins, or combinations thereof.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., an IL-12 moiety) to the external surface or the luminal surface of the EVs, e.g., exosomes.

As used herein, the term “Scaffold Y” refers to exosome proteins that were newly identified within the luminal surface of exosomes. See, e.g., International Publication No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor a moiety (e.g., an IL-12 moiety) to the lumen of the EVs, e.g., exosomes.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface and/or on the exterior surface of the EV. Similarly, in certain aspects, a functional fragment of a Scaffold Y protein retains the ability to anchor a moiety on the luminal surface of the EV. Whether a fragment is a functional fragment can be assessed by any art known methods to determine the protein content of EVs including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

In some aspects, a variant of a Scaffold Y comprises a variant having at least 70% identity to MARCKS, MARCKSL1, BASP1 or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 401 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 402 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 403 or with a functional fragment thereof. In some aspects, the variant or variant of a fragment of Scaffold Y protein retains the ability to be specifically targeted to the lumen of EVs. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “producer cell” refers to a cell used for generating an EV. A producer cell can be a cell cultured in vitro, or a cell in vivo. A producer cell includes, but not limited to, a cell known to be effective in generating EVs, e.g., exosomes, e.g., HEK293 cells, Chinese hamster ovary (CHO) cells, mesenchymal stem cells (MSCs), BJ human foreskin fibroblast cells, s9f cells, fHDF fibroblast cells, AGE.HN© neuronal precursor cells, CAP© amniocyte cells, adipose mesenchymal stem cells, and RPTEC/TERT1 cells. In certain aspects, a producer cell is an antigen-presenting cell. In some aspects, the producer cell is a bacterial cell. In some aspects, a producer cell is a dendritic cell, a B cell, a mast cell, a macrophage, a neutrophil, a Kupffer-Browicz cell, or a cell derived from any of these cells, or any combination thereof. In some aspects, the producer cell is not a bacterial cell. In other aspects, the producer cell is not an antigen-presenting cell.

As used herein the term “associated with” refers to encapsulation of a first moiety, e.g., an IL-12 moiety, into a second moiety, e.g., extracellular vesicle, or to a covalent or non-covalent bond formed between a first moiety, e.g., an IL-12 moiety, and a second moiety, e.g., extracellular vesicle, respectively. For example, in some aspects, a scaffold moiety, e.g., Scaffold X (e.g., a PTGFRN protein), is expressed in or on the extracellular vesicle and an IL-12 moiety, is loaded on the external surface of the extracellular vesicle. In one aspect, the term “associated with” means a covalent, non-peptide bond or a non-covalent bond. For example, the amino acid cysteine comprises a thiol group that can form a disulfide bond or bridge with a thiol group on a second cysteine residue. Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In other aspects, the term “associated with” means that a a first moiety, e.g., extracellular vesicle, encapsulates a second moiety, e.g., an IL-12 moiety. In some aspects, the first moiety and the second moiety can be linked to each other. In other aspects, the first moiety and the second moiety are not physically and/or chemically linked to each other.

As used herein the term “linked to” or “conjugated to” are used interchangeably and refer to a covalent or non-covalent bond formed between a first moiety and a second moiety, e.g., an IL-12 moiety and an extracellular vesicle, respectively. In some aspects, a scaffold moiety is expressed in or on the extracellular vesicle, e.g., Scaffold X (e.g., a PTGFRN protein), and an IL-12 moiety is linked to or conjugated to the portion of the Scaffold X protein (e.g., the PTGFRN protein) that is exposed on the surface of the extracellular vesicle (e.g., “surface-display of IL-12”). In some aspects, a scaffold moiety is expressed in or on the extracellular vesicle, e.g., Scaffold X (e.g., a PTGFRN protein), and an IL-12 moiety is linked to or conjugated to the portion of the Scaffold X protein (e.g., the PTGFRN protein) that is exposed to the lumen of the extracellular vesicle.

The term “loaded”, or grammatically different forms of the term (e.g., load or loaded), as used herein, refers to a status or process of having a first moiety (e.g., an IL-12 moiety) associated with a second moiety (e.g., an EV, e.g., and exosome). In some aspects, the first moiety is chemically or physically linked to the second moiety. In some aspects, the first moiety is not chemically or physically linked to the second moiety. In some aspects, the first moiety is present within the second moiety, e.g., within the lumen of an EV (e.g., an exosome), e.g., “encapsulated”. In some aspects, the first moiety is associated with the exterior surface of the second moiety, e.g., linked or conjugated to the surface of an EV (e.g., an exosome), e.g., “surface-display” of the second moiety.

The term “encapsulated”, or grammatically different forms of the term (e.g., encapsulation, or encapsulating), refers to a status or process of having a first moiety (e.g., an IL-12 moiety) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., an IL-12 moiety) into a second moiety (e.g., EVs, e.g., exosomes) are disclosed elsewhere herein.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

As used herein, the term “agonist” refers to a molecule that binds to a receptor and activates the receptor to produce a biological response. Receptors can be activated by either an endogenous or an exogenous agonist. Non-limiting examples of endogenous agonist include hormones, neurotransmitters, and cyclic dinucleotides. Non-limiting examples of exogenous agonist include drugs, small molecules, and cyclic dinucleotides. The agonist can be a full, partial, or inverse agonist.

As used herein, the term “antagonist” refers to a molecule that blocks or dampens an agonist mediated response rather than provoking a biological response itself upon bind to a receptor. Many antagonists achieve their potency by competing with endogenous ligands or substrates at structurally defined binding sites on the receptors. Non-limiting examples of antagonists include alpha blockers, beta-blocker, and calcium channel blockers. The antagonist can be a competitive, non-competitive, or uncompetitive antagonist.

The term “free IL-12 moiety” as used herein means an IL-12 moiety that is not associated with an extracellular vesicle, but otherwise identical to the IL-12 moiety associated with the extracellular vesicle. Especially when compared to an extracellular vesicle associated with an IL-12 moiety, the free IL-12 moiety is the same IL-12 moiety associated with the extracellular vesicle. In some aspects, when a free IL-12 moiety is compared to an extracellular vesicle comprising the IL-12 moiety in its efficacy, toxicity, and/or any other characteristics, the amount of the free IL-12 moiety compared to the IL-12 moiety associated with the extracellular vesicle is the same as the amount of the IL-12 moiety associated with the EV.

The term “exoIL-12” as used herein refers to an exosome loaded with an IL-12 moiety, e.g., an IL-12 polypeptide or a fragment thereof. In some aspects, the IL-12 moiety is associated with the exterior surface of the exosome (e.g., surface display of the IL-12 moiety). Non-limiting examples of exosomes comprising an IL-12 moiety can be found, for example, in U.S. Pat. No. 10,723,782 and International Publication No. WO 2019/133934 A2, each of which is incorporated by reference herein in its entirety. In some aspects, the IL-12 moiety is linked to or conjugated to the exterior surface of the exosome. In some aspects, the IL-12 moiety is linked to or conjugated to a surface exposed scaffold protein, e.g, a Scaffold X protein, e.g., a PTGFRN protein. In some aspects, the IL-12 moiety is linked to or conjugated to the lipid bilayer of the exosome. In some aspects, the exosome comprises an IL-12 moiety in the lumen of the exosome. In some aspects, the IL-12 moiety is associated with the luminal surface of the exosome, e.g., with a Scaffold protein, e.g., Scaffold X, e.g., PTGFRN. In some aspects, the IL-12 moiety is encapsulated within the lumen of the exosome and is not associated with a scaffold protein. In some aspects, the IL-12 polypeptide comprises the amino acid sequence set forth in SEQ ID NO: 13.

As used herein, the term “ligand” refers to a molecule that binds to a receptor and modulates the receptor to produce a biological response. Modulation can be activation, deactivation, blocking, or damping of the biological response mediated by the receptor. Receptors can be modulated by either an endogenous or an exogenous ligand. Non-limiting examples of endogenous ligands include antibodies and peptides. Non-limiting examples of exogenous agonist include drugs, small molecules, and cyclic dinucleotides. The ligand can be a full, partial, or inverse ligand.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function.

As used herein the term “therapeutically effective amount” is the amount of reagent or pharmaceutical compound that is sufficient to a produce a desired therapeutic effect, pharmacologic and/or physiologic effect on a subject in need thereof. A therapeutically effective amount can be a “prophylactically effective amount” as prophylaxis can be considered therapy.

As used herein, the term “pharmaceutical composition” refers to one or more of the compounds described herein, such as, e.g., an EV mixed or intermingled with, or suspended in one or more other chemical components, such as pharmaceutically-acceptable carriers and excipients. One purpose of a pharmaceutical composition is to facilitate administration of preparations of EVs to a subject. The term “excipient” or “carrier” refers to an inert substance added to a pharmaceutical composition to further facilitate administration of a compound. The term “pharmaceutically-acceptable carrier” or “pharmaceutically-acceptable excipient” and grammatical variations thereof, encompasses any of the agents approved by a regulatory agency of the US Federal government or listed in the US Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “payload” refers to a therapeutic agent that acts on a target (e.g., a target cell) that is contacted with the EV. Payloads that can be introduced into an EV and/or a producer cell include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins).

The terms “administration,” “administering,” and variants thereof refer to introducing a composition, such as an EV, or agent into a subject and includes concurrent and sequential introduction of a composition or agent. The introduction of a composition or agent into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. In some aspects, the composition is delivered to the subject by intravesical administration. Administration includes self-administration and the administration by another. A suitable route of administration allows the composition or the agent to perform its intended function. For example, if a suitable route is intravenous, the composition is administered by introducing the composition or agent into a vein of the subject.

The term “treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also include prophylaxis or prevention of a disease or condition or its symptoms thereof. In one aspect, the term “treating” or “treatment” means inducing an immune response in a subject against an antigen.

The term “prevent” or “preventing,” as used herein, refers to decreasing or reducing the occurrence or severity of a particular outcome. In some aspects, preventing an outcome is achieved through prophylactic treatment.

As used herein, the term “modulate,” “modulating”, “modify,” and/or “modulator” generally refers to the ability to alter, by increase or decrease, e.g., directly or indirectly promoting/stimulating/up-regulating or interfering with/inhibiting/down-regulating a specific concentration, level, expression, function or behavior, such as, e.g., to act as an antagonist or agonist. In some instances a modulator can increase and/or decrease a certain concentration, level, activity or function relative to a control, or relative to the average level of activity that would generally be expected or relative to a control level of activity.

As used herein, “a mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The terms “individual,” “subject,” “host,” and “patient,” are used interchangeably herein and refer to any mammalian subject for whom diagnosis, treatment, or therapy is desired, particularly humans. The methods described herein are applicable to both human therapy and veterinary applications. In some aspects, the subject is a mammal, and in other aspects the subject is a human.

As used herein, the term “substantially free” means that the sample comprising EVs comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, exogenous proteins, lipids, carbohydrates, metabolites, or a combination thereof.

A “primary tumor,” as used herein, refers to an original, or first, tumor in a subject, where the tumor initiated growth. A primary tumor is used in contrast to a “secondary tumor,” which refers to a tumor that arises after initiation of growth of the primary tumor at a location other than the location of the primary tumor, e.g., due to metastasis of cells in the primary tumor.

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

### II. Methods of Treating

Certain aspects of the present disclosure are directed to a method of preventing or treating a tumor in a subject in need thereof comprising administering to the subject a dose of an IL-12 polypeptide, wherein the amount of the IL-12 polypeptide in the dose is about 0.1 μg to about 30 μg. In some aspects, the IL-12 polypeptide is delivered by an extracellular vesicle. In some aspects, the IL-12 polypeptide is associated with an EV.

In some aspects, the tumor treatable by the present methods is a primary tumor, a secondary tumor, or both a primary tumor and a secondary tumor. In some aspects, the administering reduces the volume of a primary tumor. In some aspects, the administering is capable of reducing the volume of a primary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering.

In some aspects, the administering reduces the rate of tumor growth of a primary tumor. In some aspects, the administering is capable of reducing the growth rate of a primary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering. In some aspects, the administering is capable of ablating or stopping primary tumor growth.

The method of any one of claims 4 to 8, wherein the administering reduces the volume of a secondary tumor. In some aspects, the administering is capable of reducing the volume of a secondary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering.

In some aspects, the administering reduces the rate of tumor growth of a secondary tumor. In some aspects, the administering is capable of reducing the growth rate of a secondary tumor by at least about 1.5 fold, at least about 2 fold, at least about 3 fold, at least about 4 fold, at least about 5 fold, at least about 6 fold, at least about 7 fold, at least about 8 fold, at least about 9 fold, or at least about 10 fold compared to a monotherapy after day 14 of the administering. In some aspects, the administering is capable of ablating or stopping secondary tumor growth.

Other aspects of the disclosure are directed to methods of treating a disease or condition, e.g., a tumor, in a subject in need thereof comprising administering an extracellular vesicle (EV) comprising an interleukin 12 (IL-12) moiety, wherein the method does not comprise administering an EV comprising a STING agonist.

In some aspects, the method further comprises an anti-cancer agent. In some aspects, the anti-cancer agent comprises a checkpoint inhibitor. In some aspects, the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof. In certain aspects, the checkpoint inhibitor is an anti-PD-1 antibody.

In some aspects, the administration of the IL-12 moiety results in an objective response rate (ORR) of about 20% to about 100%, about 30% to about 100%, about 40% to about 100%, about 50% to about 100%, about 60% to about 100%, about 70% to about 100%, or about 80% to about 100%. In some aspects, the administration of the IL-12 moiety results in an ORR of about 20% to about 90%, about 20% to about 80%, about 20% to about 75%, about 20% to about 70%, about 20% to about 65%, about 20% to about 60%, about 20% to about 55%, about 20% to about 50%. In some aspects, the administration of the IL-12 moiety results in an ORR of about 25% to about 56%. In some aspects, the administration of the IL-12 moiety results in at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% ORR. In some aspects, the response rate is measured using an mSWAT analysis, e.g., for CTCL.

In some aspects, the administration of the IL-12 moiety results in a complete response in the subject. In some aspects, the administration of the IL-12 moiety results in at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% of subjects experiencing a complete response (CR). In some aspects, the administration of the IL-12 moiety results in at least about 20% of subjects experiencing a CR. In some aspects, the administration of the IL-12 moiety results in at least about 22% of subjects experiencing a CR.

Without being bound by any particular mechanism, in some aspects, administration of the IL-12 moiety disclosed herein activates immune cells that target and kill tumor cells. In some aspects, the IL-12 moiety disclosed herein, e.g., EVs loaded with an IL-12 moiety, activate T cells. In some aspects, the IL-12 moiety disclosed herein, e.g., EVs loaded with an IL-12 moiety, activate CD8+ T cells. In some aspects, the IL-12 moiety disclosed herein, e.g., EVs loaded with an IL-12 moiety, activate NK cells.

The IL-12 moiety can be administered to a subject by any route known in the art. In some aspects, the IL-12 moiety are administered parenterally, orally, intravenously (IV), intramuscularly (IM), intra-tumorally (IT), intranasally, subcutaneously, or intraperitoneally (IP). In certain aspects, the IL-12 moiety are administered directly into the tumor microenvironment, e.g., by IT delivery. In some aspects, IT delivery of the IL-12 moiety disclosed herein reduces systemic exposure, enhances selective immune cell activation, increases tissue retention (providing a prolonged response), or any combination thereof.

### II.A. Interleukin-12 (IL-12)

Interleukin 12 (IL-12) is heterodimeric cytokine produced by dendritic cells, macrophages and neutrophils. See, e.g., Interleukin-12 Signaling, Reactome, available at reactome.org/content/detail/R-HSA-9020591; and UniProtKB-P29459 (IL-12A Subunit) P29460 (IL-12B Subunit). It is encoded by the genes Interleukin-12 subunit alpha (IL12A) and Interleukin-12 subunit beta (IL12B), which encode a 35-kDa light chain (p35) and a 40-kDa heavy chain (p40), respectively. The active IL-12 heterodimer is sometimes referred to as p70. The p35 component has homology to single-chain cytokines, while p40 is homologous to the extracellular domains of members of the haematopoietic cytokine-receptor family. The IL-12 heterodimer therefore resembles a cytokine linked to a soluble receptor. IL-12 is involved in the differentiation of naïve T cells into Th1 cells and sometimes known as T cell-stimulating factor. IL-12 enhances the cytotoxic activity of Natural Killer cells and CD8+ cytotoxic T lymphocytes. IL-12 also has anti-angiogenic activity, mediated by increased production of CXCL10 via interferon gamma. Non-limiting examples IL-12 moieties usable in the present disclosure can be found, for example, in U.S. Pat. No. 10,723,782, International Publication No. WO 2019/133934 A2, and International Application No. PCT/US2020/028778, each of which is incorporated by reference herein in its entirety.

The IL-12 receptor is a heterodimer formed by Interleukin-12 receptor subunit beta-1 (IL12RB1) and Interleukin-12 receptor subunit beta-2 (IL12RB2), both of which have extensive homology to IL6ST (gpi30), the signal transducing receptor subunit of the IL6-like cytokine superfamily. IL-12RB2 is considered to play the key role in IL-12 function, in part because its expression on activated T cells is stimulated by cytokines that promote Th1 cell development and inhibited by those that promote Th2 cells development. In addition, IL-12 binding leads to IL12RB2 tyrosine phosphorylation, which provides binding sites for the kinases Non-receptor tyrosine-protein kinase TYK2 and Tyrosine-protein kinase JAK2. These activate transcription factor proteins in the Signal transducer and activator of transcription (STAT) family, particularly STAT4. is a cytokine that is produced by myeloid and other cell types.

The amino acid sequences for the IL-12 A and B subunits are shown in Table 1A.

TABLE 1A
Human IL-12A and IL-12B subunits amino acid sequences.

| Human IL-12A Subunit (UniProtKB - P29459) | Human IL-12B Subunit (UniProtKB - P29460) |
|---|---|
| (signal peptide) | (signal peptide) |
| MCPARSLLLVATLVLLDHLSLARNLPVATPDPGMFPCLH | MCHQQLVISWFSLVFLASPLVAIWELKKDVYVVELDWY |
| HSQNLLRAVSNMLQKARQTLEFYPCTSEEIDHEDITKDK | PDAPGEMVVLTCDTPEEDGITWTLDQSSEVLGSGKTLTI |
| TSTVEACLPLELTKNESCLNSRETSFITNGSCLASRKTSFM | QVKEFGDAGQYTCHKGGEVLSHSLLLLHKKEDGIWSTD |
| MALCLSSIYEDLKMYQVEFKTMNAKLLMDPKRQIFLDQ | ILKDQKEPKNKTFLRCEAKNYSGRFTCWWLTTISTDLTFS |
| NMLAVIDELMQALNFNSETVPQKSSLEEPDFYKTKIKLCI | VKSSRGSSDPQGVTCGAATLSAERVRGDNKEYEYSVEC |
| LLHAFRIRAVTIDRVMSYLNAS | QEDSACPAAEESLPIEVMVDAVHKLKYENYTSSFFIRDII |
| SEQ ID NO: 11 | KPDPPKNLQLKPLKNSRQVEVSWEYPDTWSTPHSYFSL |
| | TFCVQVQGKSKREKKDRVFTDKTSATVICRKNASISVRA |
| | QDRYYSSSWSEWASVPCS |
| | SEQ ID NO: 12 |

In some aspects, the IL-12 moiety comprises an IL-12 polypeptide. In some aspects, the IL-12 moiety comprises an IL-12 protein. In some aspects, the IL-12 polypeptide comprises full length human IL-12, e.g., IL-12 heterodimer. In some aspects, the IL-12 heterodimer comprises a fusion protein, wherein the IL-12 alpha subunit is covalently linked to the IL-12 beta subunit (SEQ ID NO: 13; Table 1). In some aspects, the IL-12 moiety comprises the IL-12 alpha subunit. In some aspects, the IL-12 moiety comprises the IL-12 beta subunit.

TABLE 1B
Human IL-12A-IL-12B fusion amino acid sequence.

| | |
|---|---|
| IL-12 | MCHQQLVISWFSLVFLASPLVAIWELKKDVYVVELDWYPDAPGEMVVLTCDTPEED |
| Fusion | GITWTLDQSSEVLGSGKTLTIQVKEFGDAGQYTCHKGGEVLSHSLLLLHKKEDGIW |
| (signal | STDILKDQKEPKNKTFLRCEAKNYSGRFTCWWLTTISTDLTFSVKSSRGSSDPQGV |
| peptide- | TCGAATLSAERVRGDNKEYEYSVECQEDSACPAAEESLPIEVMVDAVHKLKYENYT |
| p40- | SSFFIRDIIKPDPPKNLQLKPLKNSRQVEVSWEYPDTWSTPHSYFSLTFCVQVQGK |
| linker- | SKREKKDRVFTDKTSATVICRKNASISVRAQDRYYSSSWSEWASVPCSGGSGGGSG |
| p35) | GGGSGGGGSGGGSGGRNLPVATPDPGMFPCLHHSQNLLRAVSNMLQKARQTLEFYP |
| SEQ ID | CTSEEIDHEDITKDKTSTVEACLPLELTKNESCLNSRETSFITNGSCLASRKTSFM |
| NO: 13 | MALCLSSIYEDLKMYQVEFKTMNAKLLMDPKRQIFLDQNMLAVIDELMQALNFNSE |
| | TVPQKSSLEEPDFYKTKIKLCILLHAFRIRAVTIDRVMSYLNAS |

In some aspects, the IL-12 heterodimer comprises an IL-12 alpha subunit covalently linked to an IL-12 beta subunit by a linker. In some aspects, the linker comprises one or more amino acids. In some aspects, the linker is a linker disclosed herein. In some aspect, the linker comprises a Gly/Ser linker. In some aspects, the linker is a cleavable linker. In some aspects, the linker comprises disulfide bond.

In some aspects, the IL-12 moiety comprises a molecule having IL-12 activity. In some aspects, the molecule having IL-12 activity is an IL-12 analog. In some aspects, the molecule having IL-12 activity comprises a molecule which activates IL-12 receptor.

In some aspects, the IL-12 moiety comprises a nucleic acid molecule encoding an IL-12 protein, e.g., an IL-12 alpha subunit, an IL-12 beta subunit, and/or an IL-12 heterodimer. In some aspects, the nucleic acid molecule encodes an IL-12 alpha subunit. In some aspects, the nucleic acid molecule encodes an IL-12 beta subunit. In some aspects, the nucleic acid molecule encodes an IL-12 alpha subunit and an IL-12 beta subunit. In some aspects, the nucleic acid molecule encodes an IL-12 alpha subunit covalently linked to an IL-12 beta subunit. In some aspects, the nucleic acid molecule encodes an IL-12 heterodimer.

In some aspects, the IL-12 moiety comprises a nucleic acid molecule, wherein the nucleic acid molecule is packed in a vector. In some aspects, the vector is a viral vector. In some aspects, the vector is based on a DNA virus, such as adenovirus, adeno-associated virus (AAV) and herpes virus, as well as retroviral based vectors. In some aspects, the vector is a lentivirus. In some aspects, the virus is an AAV.

In some aspects, the IL-12 moiety comprises a p35 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 11. In some aspects, the IL-12 comprises an amino acid sequence having at least about 90% sequence identity to SEQ ID NO: 11. In some aspects, the IL-12 comprises an amino acid sequence having at least about 95% sequence identity to SEQ ID NO: 11. In some aspects, the IL-12 comprises an amino acid sequence having at least about 96% sequence identity to SEQ ID NO: 11. In some aspects, the IL-12 comprises an amino acid sequence having at least about 97% sequence identity to SEQ ID NO: 11. In some aspects, the IL-12 comprises an amino acid sequence having at least about 98% sequence identity to SEQ ID NO: 11. In some aspects, the IL-12 comprises an amino acid sequence having at least about 99% sequence identity to SEQ ID NO: 11. In some aspects, the IL-12 comprises the amino acid sequence set forth in SEQ ID NO: 11. In some aspects, the IL-12 moiety lacks a signal peptide (see Table 1A).

In some aspects, the IL-12 moiety comprises a p40 polypeptide or a fragment thereof. In some aspects, the IL-12 comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 12. In some aspects, the IL-12 comprises an amino acid sequence having at least about 90% sequence identity to SEQ ID NO: 12. In some aspects, the IL-12 comprises an amino acid sequence having at least about 95% sequence identity to SEQ ID NO: 12. In some aspects, the IL-12 comprises an amino acid sequence having at least about 96% sequence identity to SEQ ID NO: 12. In some aspects, the IL-12 comprises an amino acid sequence having at least about 97% sequence identity to SEQ ID NO: 12. In some aspects, the IL-12 comprises an amino acid sequence having at least about 98% sequence identity to SEQ ID NO: 12. In some aspects, the IL-12 comprises an amino acid sequence having at least about 99% sequence identity to SEQ ID NO: 12. In some aspects, the IL-12 comprises the amino acid sequence set forth in SEQ ID NO: 12. In some aspects, the IL-12 moiety lacks a signal peptide (see Table 1A).

In some aspects, the IL-12 moiety comprises a p35 polypeptide or a fragment thereof and a p40 polypeptide or a fragment thereof. In some aspects, IL-12 moiety comprises a single polypeptide, wherein the p35 polypeptide or a fragment thereof is linked to the p40 polypeptide or a fragment thereof. In some aspects, the p35 polypeptide or a fragment thereof is linked to the p40 polypeptide or a fragment thereof by a linker. In some aspects, the linker is a peptide linker. In some aspects, the linker comprises one or more amino acids. In some aspects, the linker comprises a Gly-Ser (GS) linker. In some aspects, the GS linker comprises (G₄S)ₙ, wherein n is an integer between 1 and 10. In some aspects, the GS linker comprises (G₃S)ₙ, wherein n is an integer between 1 and 10.

In certain aspects, the IL-12 moiety comprises an amino acid sequence having at least about 50%, at least about 60%, at least about 70%, at least about 80%, at least about 85%, at least about 90%, at least about 95%, at least about 96%, at least about 97%, at least about 98%, at least about 99%, or about 100% sequence identity to SEQ ID NO: 13. In some aspects, the IL-12 comprises an amino acid sequence having at least about 90% sequence identity to SEQ ID NO: 13. In some aspects, the IL-12 comprises an amino acid sequence having at least about 95% sequence identity to SEQ ID NO: 13. In some aspects, the IL-12 comprises an amino acid sequence having at least about 96% sequence identity to SEQ ID NO: 13. In some aspects, the IL-12 comprises an amino acid sequence having at least about 97% sequence identity to SEQ ID NO: 13. In some aspects, the IL-12 comprises an amino acid sequence having at least about 98% sequence identity to SEQ ID NO: 13. In some aspects, the IL-12 comprises an amino acid sequence having at least about 99% sequence identity to SEQ ID NO: 13. In some aspects, the IL-12 comprises the amino acid sequence set forth in SEQ ID NO: 13. In some aspects, the IL-12 consists of the amino acid sequence set forth in SEQ ID NO: 13. In some aspects, the IL-12 consists essentially of the amino acid sequence set forth in SEQ ID NO: 13. In some aspects, the IL-12 moiety lacks a signal peptide (see Table 1B).

### II.B. Dosing

In some aspects, the method comprises administering to a subject a dose of an IL-12 polypeptide, wherein the amount of the IL-12 polypeptide in the dose is about 0.1 μg to about 30 μg. In some aspects, the amount of the IL-12 polypeptide is measured using a fluorescence assay. In some aspects, the amount of the IL-12 polypeptide is measured using IL-12 AlphaLISA. In some aspects, the dose of the IL-12 polypeptide administered is based on the weight of the IL-12 polypeptide, without including the weight of an anchoring moiety, scaffold moiety, or other linker that may be used to associate the IL-12 polypeptide with the exosome.

In some aspects, the dose comprises at least about 0.1 μg, at least about 0.2 μg, at least about 0.3 μg, at least about 0.4 μg, at least about 0.5 μg, at least about 0.6 μg, at least about 0.7 μg, at least about 0.8 μg, at least about 0.9 μg, at least about 1.0 μg, at least about 1.1 μg, at least about 1.2 μg, at least about 1.3 μg, at least about 1.4 μg, at least about 1.5 μg, at least about 1.6 μg, at least about 1.7 μg, at least about 1.8 μg, at least about 1.9 μg, at least about 2.0 μg, at least about 2.1 μg, at least about 2.2 μg, at least about 2.3 μg, at least about 2.4 μg, at least about 2.5 μg, at least about 2.6 μg, at least about 2.7 μg, at least about 2.8 μg, at least about 2.9 μg, at least about 3.0 μg, at least about 3.1 μg, at least about 3.2 μg, at least about 3.3 μg, at least about 3.4 μg, at least about 3.5 μg, at least about 3.6 μg, at least about 3.7 μg, at least about 3.8 μg, at least about 3.9 μg, at least about 4.0 μg, at least about 4.1 μg, at least about 4.2 μg, at least about 4.3 μg, at least about 4.4 μg, at least about 4.5 μg, at least about 4.6 μg, at least about 4.7 μg, at least about 4.8 μg, at least about 4.9 μg, at least about 5.0 μg, at least about 5.1 μg, at least about 5.2 μg, at least about 5.3 μg, at least about 5.4 μg, at least about 5.5 μg, at least about 5.6 μg, at least about 5.7 μg, at least about 5.8 μg, at least about 5.9 μg, at least about 6.0 μg, at least about 6.5 μg, at least about 7.0 μg, at least about 7.5 μg, at least about 8.0 μg, at least about 8.5 μg, at least about 9.0 μg, at least about 9.5 μg, at least about 10.0 μg, at least about 10.5 μg, at least about 11.0 μg, at least about 11.5 μg, at least about 12.0 μg, at least about 12.5 μg, at least about 13.0 μg, at least about 14 μg, at least about 15 μg, at least about 16 μg, at least about 17 μg, at least about 18 μg, at least about 19 μg, at least about 20 μg, at least about 25 μg, or at least about 30 μg of the IL-12 polypeptide.

In some aspects, the dose comprises at least about 0.3 μg of the IL-12 polypeptide. In some aspects, the dose comprises at least about 0.4 μg of the IL-12 polypeptide. In some aspects, the dose comprises at least about 0.5 μg of the IL-12 polypeptide. In some aspects, the dose comprises at least about 0.6 μg of the IL-12 polypeptide. In some aspects, the dose comprises at least about 0.7 μg of the IL-12 polypeptide. In some aspects, the dose comprises at least about 0.8 μg of the IL-12 polypeptide. In some aspects, the dose comprises at least about 0.9 μg of the IL-12 polypeptide. In some aspects, the dose comprises at least about 1.0 μg of the IL-12 polypeptide. In some aspects, the dose comprises at least about 1.5 μg of the IL-12 polypeptide. In some aspects, the dose comprises at least about 2.0 μg of the IL-12 polypeptide. In some aspects, the dose comprises at least about 2.5 μg of the IL-12 polypeptide. In some aspects, the dose comprises at least about 3.0 μg of the IL-12 polypeptide. In some aspects, the dose comprises at least about 3.5 μg of the IL-12 polypeptide. In some aspects, the dose comprises at least about 4.0 μg of the IL-12 polypeptide. In some aspects, the dose comprises at least about 4.5 μg of the IL-12 polypeptide. In some aspects, the dose comprises at least about 5.0 μg of the IL-12 polypeptide. In some aspects, the dose comprises at least about 5.5 μg of the IL-12 polypeptide. In some aspects, the dose comprises at least about 6.0 μg of the IL-12 polypeptide. In some aspects, the dose comprises at least about 6.5 μg of the IL-12 polypeptide. In some aspects, the dose comprises at least about 7.0 μg of the IL-12 polypeptide. In some aspects, the dose comprises at least about 7.5 μg of the IL-12 polypeptide. In some aspects, the dose comprises at least about 8.0 μg of the IL-12 polypeptide. In some aspects, the dose comprises at least about 8.5 μg of the IL-12 polypeptide. In some aspects, the dose comprises at least about 9.0 μg of the IL-12 polypeptide. In some aspects, the dose comprises at least about 9.5 μg of the IL-12 polypeptide. In some aspects, the dose comprises at least about 10.0 μg of the IL-12 polypeptide. In some aspects, the dose comprises at least about 10.5 μg of the IL-12 polypeptide. In some aspects, the dose comprises at least about 11.0 μg of the IL-12 polypeptide. In some aspects, the dose comprises at least about 11.5 μg of the IL-12 polypeptide. In some aspects, the dose comprises at least about 12.0 μg of the IL-12 polypeptide. In some aspects, the dose comprises at least about 12.5 μg of the IL-12 polypeptide. In some aspects, the dose comprises at least about 13.0 μg of the IL-12 polypeptide. In some aspects, the dose comprises at least about 13.5 μg of the IL-12 polypeptide. In some aspects, the dose comprises at least about 14.0 μg of the IL-12 polypeptide. In some aspects, the dose comprises at least about 14.5 μg of the IL-12 polypeptide. In some aspects, the dose comprises at least about 15 μg of the IL-12 polypeptide. In some aspects, the dose comprises at least about 16 μg of the IL-12 polypeptide. In some aspects, the dose comprises at least about 17 μg of the IL-12 polypeptide. In some aspects, the dose comprises at least about 18 μg of the IL-12 polypeptide. In some aspects, the dose comprises at least about 19 μg of the IL-12 polypeptide. In some aspects, the dose comprises at least about 20 μg of the IL-12 polypeptide. In some aspects, the dose comprises at least about 21 μg of the IL-12 polypeptide. In some aspects, the dose comprises at least about 22 μg of the IL-12 polypeptide. In some aspects, the dose comprises at least about 23 μg of the IL-12 polypeptide. In some aspects, the dose comprises at least about 24 μg of the IL-12 polypeptide. In some aspects, the dose comprises at least about 25 μg of the IL-12 polypeptide. In some aspects, the dose comprises at least about 26 μg of the IL-12 polypeptide. In some aspects, the dose comprises at least about 27 μg of the IL-12 polypeptide. In some aspects, the dose comprises at least about 28 μg of the IL-12 polypeptide. In some aspects, the dose comprises at least about 29 μg of the IL-12 polypeptide. In some aspects, the dose comprises at least about 30 μg of the IL-12 polypeptide.

In some aspects, the dose of the IL-12 polypeptide is administered at least two times, at least three times, at least four times, at least five times, or at least six times. In some aspects, the dose of the IL-12 polypeptide is administered once about every week, once about every two weeks, once about every three weeks, once about every four weeks, once about every six weeks, or once about every eight weeks. In some aspects, the IL-12 polypeptide is administered about once per week. In some aspects, the IL-12 polypeptide is administered once about every two weeks. In some aspects, the IL-12 polypeptide is administered once about every three weeks. In some aspects, the IL-12 polypeptide is administered once about every four weeks.

In some aspects, the dose is administered once about every week, once about every other week, once about every three weeks, or once about every four weeks. In some aspects, the dose is administered once about every other week. In some aspects, the dose is administered once about every 7 to 21 days, once about every 7 to 18 days, once about every 7 to 14 days, once about every 10 to 21 days, once about every 10 to 18 days, once about every 10 to 14 days, once about every 10 to 16 days, once about every 12 to about 20 days, once about every 12 to about 18 days, once about every 12 to about 16 days, once about every 12 to about 14 days, once about every 14 to about 21 days, once about every 14 to about 16 days, once about every 14 to about 18 days, or once about every 14 to about 21 days. In some aspects, the dose is administered once about every 7 days. In some aspects, the dose is administered once about every 10 days. In some aspects, the dose is administered once about every 12 days. In some aspects, the dose is administered once about every 14 days. In some aspects, the dose is administered once about every 16 days. In some aspects, the dose is administered once about every 18 days. In some aspects, the dose is administered once about every 20 days. In some aspects, the dose is administered once about every 21 days.

In some aspects, the dose of the IL-12 polypeptide is administered at a therapeutically effective amount between about 5 μg and about 7 μg of the IL-12 polypeptide, e.g., about 6 μg of the IL-12 polypeptide, once about every week, once about every other week, once about every three weeks, or once about every four weeks. In some aspects, the dose of the IL-12 polypeptide is administered at a therapeutically effective amount between about 5 μg and about 7 μg of the IL-12 polypeptide, e.g., about 6 μg of the IL-12 polypeptide, once about every other week. In some aspects, the dose of the IL-12 polypeptide is administered at a therapeutically effective amount between about 5 μg and about 7 μg of the IL-12 polypeptide, e.g., about 6 μg of the IL-12 polypeptide, once about every 10 to 18 days, once about every 12 to about 16 days, once about every 14 to about 21 days, once about every 10 to 14 days, or once about every 14 to about 18 days. In some aspects, the dose of the IL-12 polypeptide is administered at a therapeutically effective amount between about 5 μg and about 7 μg of the IL-12 polypeptide, e.g., about 6 μg of the IL-12 polypeptide, once about every 14 days.

In some aspects, the dose of the IL-12 polypeptide is administered at a therapeutically effective amount of about 3.0 μg of the IL-12 polypeptide once about every week. In some aspects, the dose of the IL-12 polypeptide is administered at a therapeutically effective amount of about 3.0 μg of the IL-12 polypeptide once about every 7 days. In some aspects, the dose of the IL-12 polypeptide is administered at a therapeutically effective amount of about 3.0 μg of the IL-12 polypeptide once about every 8 days. In some aspects, the dose of the IL-12 polypeptide is administered at a therapeutically effective amount of about 3.0 μg of the IL-12 polypeptide once about every 9 days. In some aspects, the dose of the IL-12 polypeptide is administered at a therapeutically effective amount of about 3.0 μg of the IL-12 polypeptide once about every 10 days. In some aspects, the dose of the IL-12 polypeptide is administered at a therapeutically effective amount of about 3.0 μg of the IL-12 polypeptide once about every 11 days. In some aspects, the dose of the IL-12 polypeptide is administered at a therapeutically effective amount of about 3.0 μg of the IL-12 polypeptide once about every 12 days. In some aspects, the dose of the IL-12 polypeptide is administered at a therapeutically effective amount of about 3.0 μg of the IL-12 polypeptide once about every 13 days. In some aspects, the dose of the IL-12 polypeptide is administered at a therapeutically effective amount of about 3.0 μg of the IL-12 polypeptide once about every 14 days. In some aspects, the dose of the IL-12 polypeptide is administered at a therapeutically effective amount of about 3.0 μg of the IL-12 polypeptide once about every 2 weeks. In some aspects, the dose of the IL-12 polypeptide is administered at a therapeutically effective amount of about 3.0 μg of the IL-12 polypeptide once about every 15 days. In some aspects, the dose of the IL-12 polypeptide is administered at a therapeutically effective amount of about 3.0 μg of the IL-12 polypeptide once about every 15 days. In some aspects, the dose of the IL-12 polypeptide is administered at a therapeutically effective amount of about 3.0 μg of the IL-12 polypeptide once about every 17 days. In some aspects, the dose of the IL-12 polypeptide is administered at a therapeutically effective amount of about 3.0 μg of the IL-12 polypeptide once about every 18 days. In some aspects, the dose of the IL-12 polypeptide is administered at a therapeutically effective amount of about 3.0 μg of the IL-12 polypeptide once about every 19 days. In some aspects, the dose of the IL-12 polypeptide is administered at a therapeutically effective amount of about 3.0 μg of the IL-12 polypeptide once about every 20 days. In some aspects, the dose of the IL-12 polypeptide is administered at a therapeutically effective amount of about 3.0 μg of the IL-12 polypeptide once about every 21 days. In some aspects, the dose of the IL-12 polypeptide is administered at a therapeutically effective amount of about 3.0 μg of the IL-12 polypeptide once about every 3 weeks. In some aspects, the dose of the IL-12 polypeptide is administered at a therapeutically effective amount of about 3.0 μg of the IL-12 polypeptide once about every week. In some aspects, the dose of the IL-12 polypeptide is administered at a therapeutically effective amount of about 3.0 μg of the IL-12 polypeptide once about every 7 days. In some aspects, the dose of the IL-12 polypeptide is administered at a therapeutically effective amount of about 3.0 μg of the IL-12 polypeptide once about every 8 days. In some aspects, the dose of the IL-12 polypeptide is administered at a therapeutically effective amount of about 3.0 μg of the IL-12 polypeptide once about every 9 days. In some aspects, the dose of the IL-12 polypeptide is administered at a therapeutically effective amount of about 3.0 μg of the IL-12 polypeptide once about every 10 days. In some aspects, the dose of the IL-12 polypeptide is administered at a therapeutically effective amount of about 3.0 μg of the IL-12 polypeptide once about every 11 days. In some aspects, the dose of the IL-12 polypeptide is administered at a therapeutically effective amount of about 3.0 μg of the IL-12 polypeptide once about every 12 days. In some aspects, the dose of the IL-12 polypeptide is administered at a therapeutically effective amount of about 3.0 μg of the IL-12 polypeptide once about every 13 days. In some aspects, the dose of the IL-12 polypeptide is administered at a therapeutically effective amount of about 3.0 μg of the IL-12 polypeptide once about every 14 days. In some aspects, the dose of the IL-12 polypeptide is administered at a therapeutically effective amount of about 3.0 μg of the IL-12 polypeptide once about every 2 weeks. In some aspects, the dose of the IL-12 polypeptide is administered at a therapeutically effective amount of about 3.0 μg of the IL-12 polypeptide once about every 15 days. In some aspects, the dose of the IL-12 polypeptide is administered at a therapeutically effective amount of about 3.0 μg of the IL-12 polypeptide once about every 15 days. In some aspects, the dose of the IL-12 polypeptide is administered at a therapeutically effective amount of about 3.0 μg of the IL-12 polypeptide once about every 17 days. In some aspects, the dose of the IL-12 polypeptide is administered at a therapeutically effective amount of about 3.0 μg of the IL-12 polypeptide once about every 18 days. In some aspects, the dose of the IL-12 polypeptide is administered at a therapeutically effective amount of about 3.0 μg of the IL-12 polypeptide once about every 19 days. In some aspects, the dose of the IL-12 polypeptide is administered at a therapeutically effective amount of about 3.0 μg of the IL-12 polypeptide once about every 20 days. In some aspects, the dose of the IL-12 polypeptide is administered at a therapeutically effective amount of about 3.0 μg of the IL-12 polypeptide once about every 21 days. In some aspects, the dose of the IL-12 polypeptide is administered at a therapeutically effective amount of about 3.0 μg of the IL-12 polypeptide once about every 3 weeks.

In some aspects, the dose of the IL-12 polypeptide is administered at a therapeutically effective amount of about 6.0 μg of the IL-12 polypeptide once about every week. In some aspects, the dose of the IL-12 polypeptide is administered at a therapeutically effective amount of about 6.0 μg of the IL-12 polypeptide once about every 7 days. In some aspects, the dose of the IL-12 polypeptide is administered at a therapeutically effective amount of about 6.0 μg of the IL-12 polypeptide once about every 8 days. In some aspects, the dose of the IL-12 polypeptide is administered at a therapeutically effective amount of about 6.0 μg of the IL-12 polypeptide once about every 9 days. In some aspects, the dose of the IL-12 polypeptide is administered at a therapeutically effective amount of about 6.0 μg of the IL-12 polypeptide once about every 10 days. In some aspects, the dose of the IL-12 polypeptide is administered at a therapeutically effective amount of about 6.0 μg of the IL-12 polypeptide once about every 11 days. In some aspects, the dose of the IL-12 polypeptide is administered at a therapeutically effective amount of about 6.0 μg of the IL-12 polypeptide once about every 12 days. In some aspects, the dose of the IL-12 polypeptide is administered at a therapeutically effective amount of about 6.0 μg of the IL-12 polypeptide once about every 13 days. In some aspects, the dose of the IL-12 polypeptide is administered at a therapeutically effective amount of about 6.0 μg of the IL-12 polypeptide once about every 14 days. In some aspects, the dose of the IL-12 polypeptide is administered at a therapeutically effective amount of about 6.0 μg of the IL-12 polypeptide once about every 2 weeks. In some aspects, the dose of the IL-12 polypeptide is administered at a therapeutically effective amount of about 6.0 μg of the IL-12 polypeptide once about every 15 days. In some aspects, the dose of the IL-12 polypeptide is administered at a therapeutically effective amount of about 6.0 μg of the IL-12 polypeptide once about every 15 days. In some aspects, the dose of the IL-12 polypeptide is administered at a therapeutically effective amount of about 6.0 μg of the IL-12 polypeptide once about every 17 days. In some aspects, the dose of the IL-12 polypeptide is administered at a therapeutically effective amount of about 6.0 μg of the IL-12 polypeptide once about every 18 days. In some aspects, the dose of the IL-12 polypeptide is administered at a therapeutically effective amount of about 6.0 μg of the IL-12 polypeptide once about every 19 days. In some aspects, the dose of the IL-12 polypeptide is administered at a therapeutically effective amount of about 6.0 μg of the IL-12 polypeptide once about every 20 days. In some aspects, the dose of the IL-12 polypeptide is administered at a therapeutically effective amount of about 6.0 μg of the IL-12 polypeptide once about every 21 days. In some aspects, the dose of the IL-12 polypeptide is administered at a therapeutically effective amount of about 6.0 μg of the IL-12 polypeptide once about every 3 weeks. In some aspects, the dose of the IL-12 polypeptide is administered at a therapeutically effective amount of about 6.0 μg of the IL-12 polypeptide once about every week. In some aspects, the dose of the IL-12 polypeptide is administered at a therapeutically effective amount of about 6.0 μg of the IL-12 polypeptide once about every 7 days. In some aspects, the dose of the IL-12 polypeptide is administered at a therapeutically effective amount of about 6.0 μg of the IL-12 polypeptide once about every 8 days. In some aspects, the dose of the IL-12 polypeptide is administered at a therapeutically effective amount of about 6.0 μg of the IL-12 polypeptide once about every 9 days. In some aspects, the dose of the IL-12 polypeptide is administered at a therapeutically effective amount of about 6.0 μg of the IL-12 polypeptide once about every 10 days. In some aspects, the dose of the IL-12 polypeptide is administered at a therapeutically effective amount of about 6.0 μg of the IL-12 polypeptide once about every 11 days. In some aspects, the dose of the IL-12 polypeptide is administered at a therapeutically effective amount of about 6.0 μg of the IL-12 polypeptide once about every 12 days. In some aspects, the dose of the IL-12 polypeptide is administered at a therapeutically effective amount of about 6.0 μg of the IL-12 polypeptide once about every 13 days. In some aspects, the dose of the IL-12 polypeptide is administered at a therapeutically effective amount of about 6.0 μg of the IL-12 polypeptide once about every 14 days. In some aspects, the dose of the IL-12 polypeptide is administered at a therapeutically effective amount of about 6.0 μg of the IL-12 polypeptide once about every 2 weeks. In some aspects, the dose of the IL-12 polypeptide is administered at a therapeutically effective amount of about 6.0 μg of the IL-12 polypeptide once about every 15 days. In some aspects, the dose of the IL-12 polypeptide is administered at a therapeutically effective amount of about 6.0 μg of the IL-12 polypeptide once about every 15 days. In some aspects, the dose of the IL-12 polypeptide is administered at a therapeutically effective amount of about 6.0 μg of the IL-12 polypeptide once about every 17 days. In some aspects, the dose of the IL-12 polypeptide is administered at a therapeutically effective amount of about 6.0 μg of the IL-12 polypeptide once about every 18 days. In some aspects, the dose of the IL-12 polypeptide is administered at a therapeutically effective amount of about 6.0 μg of the IL-12 polypeptide once about every 19 days. In some aspects, the dose of the IL-12 polypeptide is administered at a therapeutically effective amount of about 6.0 μg of the IL-12 polypeptide once about every 20 days. In some aspects, the dose of the IL-12 polypeptide is administered at a therapeutically effective amount of about 6.0 μg of the IL-12 polypeptide once about every 21 days. In some aspects, the dose of the IL-12 polypeptide is administered at a therapeutically effective amount of about 6.0 μg of the IL-12 polypeptide once about every 3 weeks.

In some aspects, the dose of the IL-12 polypeptide is administered at a therapeutically effective amount of about 12.0 μg of the IL-12 polypeptide once about every week. In some aspects, the dose of the IL-12 polypeptide is administered at a therapeutically effective amount of about 12.0 μg of the IL-12 polypeptide once about every 7 days. In some aspects, the dose of the IL-12 polypeptide is administered at a therapeutically effective amount of about 12.0 μg of the IL-12 polypeptide once about every 8 days. In some aspects, the dose of the IL-12 polypeptide is administered at a therapeutically effective amount of about 12.0 μg of the IL-12 polypeptide once about every 9 days. In some aspects, the dose of the IL-12 polypeptide is administered at a therapeutically effective amount of about 12.0 μg of the IL-12 polypeptide once about every 10 days. In some aspects, the dose of the IL-12 polypeptide is administered at a therapeutically effective amount of about 12.0 μg of the IL-12 polypeptide once about every 11 days. In some aspects, the dose of the IL-12 polypeptide is administered at a therapeutically effective amount of about 12.0 μg of the IL-12 polypeptide once about every 12 days. In some aspects, the dose of the IL-12 polypeptide is administered at a therapeutically effective amount of about 12.0 μg of the IL-12 polypeptide once about every 13 days. In some aspects, the dose of the IL-12 polypeptide is administered at a therapeutically effective amount of about 12.0 μg of the IL-12 polypeptide once about every 14 days. In some aspects, the dose of the IL-12 polypeptide is administered at a therapeutically effective amount of about 12.0 μg of the IL-12 polypeptide once about every 2 weeks. In some aspects, the dose of the IL-12 polypeptide is administered at a therapeutically effective amount of about 12.0 μg of the IL-12 polypeptide once about every 15 days. In some aspects, the dose of the IL-12 polypeptide is administered at a therapeutically effective amount of about 12.0 μg of the IL-12 polypeptide once about every 15 days. In some aspects, the dose of the IL-12 polypeptide is administered at a therapeutically effective amount of about 12.0 μg of the IL-12 polypeptide once about every 17 days. In some aspects, the dose of the IL-12 polypeptide is administered at a therapeutically effective amount of about 12.0 μg of the IL-12 polypeptide once about every 18 days. In some aspects, the dose of the IL-12 polypeptide is administered at a therapeutically effective amount of about 12.0 μg of the IL-12 polypeptide once about every 19 days. In some aspects, the dose of the IL-12 polypeptide is administered at a therapeutically effective amount of about 12.0 μg of the IL-12 polypeptide once about every 20 days. In some aspects, the dose of the IL-12 polypeptide is administered at a therapeutically effective amount of about 12.0 μg of the IL-12 polypeptide once about every 21 days. In some aspects, the dose of the IL-12 polypeptide is administered at a therapeutically effective amount of about 12.0 μg of the IL-12 polypeptide once about every 3 weeks. In some aspects, the dose of the IL-12 polypeptide is administered at a therapeutically effective amount of about 12.0 μg of the IL-12 polypeptide once about every 22 days. In some aspects, the dose of the IL-12 polypeptide is administered at a therapeutically effective amount of about 12.0 μg of the IL-12 polypeptide once about every 23 days. In some aspects, the dose of the IL-12 polypeptide is administered at a therapeutically effective amount of about 12.0 μg of the IL-12 polypeptide once about every 24 days. In some aspects, the dose of the IL-12 polypeptide is administered at a therapeutically effective amount of about 12.0 μg of the IL-12 polypeptide once about every 25 days. In some aspects, the dose of the IL-12 polypeptide is administered at a therapeutically effective amount of about 12.0 μg of the IL-12 polypeptide once about every 26 days. In some aspects, the dose of the IL-12 polypeptide is administered at a therapeutically effective amount of about 12.0 μg of the IL-12 polypeptide once about every 27 days. In some aspects, the dose of the IL-12 polypeptide is administered at a therapeutically effective amount of about 12.0 μg of the IL-12 polypeptide once about every 28 days. In some aspects, the dose of the IL-12 polypeptide is administered at a therapeutically effective amount of about 12.0 μg of the IL-12 polypeptide once about every 4 weeks. In some aspects, the dose of the IL-12 polypeptide is administered at a therapeutically effective amount of about 12.0 μg of the IL-12 polypeptide once about every month.

In some aspects, the dose of the IL-12 polypeptide is administered at a therapeutically effective amount of about 3.0 μg of the IL-12 polypeptide once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the dose of the IL-12 polypeptide is administered at a therapeutically effective amount of about 3.0 μg of the IL-12 polypeptide once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the dose of the IL-12 polypeptide is administered at a therapeutically effective amount of about 3.0 μg of the IL-12 polypeptide once about every 2 weeks for at least about 6 weeks. In some aspects, the dose of the IL-12 polypeptide is administered at a therapeutically effective amount of about 3.0 μg of the IL-12 polypeptide once about every 2 weeks for at least about 12 weeks. In some aspects, the dose of the IL-12 polypeptide is administered at a therapeutically effective amount of about 3.0 μg of the IL-12 polypeptide once about every 2 weeks for as long as therapeutically effective.

In some aspects, the dose of the IL-12 polypeptide is administered at a therapeutically effective amount of about 6.0 μg of the IL-12 polypeptide once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the dose of the IL-12 polypeptide is administered at a therapeutically effective amount of about 6.0 μg of the IL-12 polypeptide once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the dose of the IL-12 polypeptide is administered at a therapeutically effective amount of about 6.0 μg of the IL-12 polypeptide once about every 2 weeks for at least about 6 weeks. In some aspects, the dose of the IL-12 polypeptide is administered at a therapeutically effective amount of about 6.0 μg of the IL-12 polypeptide once about every 2 weeks for at least about 12 weeks. In some aspects, the dose of the IL-12 polypeptide is administered at a therapeutically effective amount of about 6.0 μg of the IL-12 polypeptide once about every 2 weeks for as long as therapeutically effective.

In some aspects, the dose of the IL-12 polypeptide is administered at a therapeutically effective amount of about 12.0 μg of the IL-12 polypeptide once about every 2 weeks for at least about 1 dose, at least about 2 doses, at least about 3 doses, at least about 4 doses, at least about 5 doses, at least about 6 doses, at least about 7 doses, at least about 8 doses, at least about 9 doses, at least about 10 doses, at least about 11 doses, at least about 12 doses, at least about 13 doses, at least about 14 doses, at least about 15 doses, at least about 16 doses, at least about 17 doses, at least about 18 doses, at least about 19 doses, at least about 20 doses, at least about 25 doses, at least about 30 doses, at least about 35 doses, at least about 40 doses, at least about 45 doses, or at least about 50 doses. In some aspects, the dose of the IL-12 polypeptide is administered at a therapeutically effective amount of about 12.0 μg of the IL-12 polypeptide once about every 2 weeks for at least about 2 weeks, at least about 4 weeks, at least about weeks, at least about 6 weeks, at least about 8 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, or for at least about 52 weeks. In some aspects, the dose of the IL-12 polypeptide is administered at a therapeutically effective amount of about 12.0 μg of the IL-12 polypeptide once about every 2 weeks for at least about 6 weeks. In some aspects, the dose of the IL-12 polypeptide is administered at a therapeutically effective amount of about 12.0 μg of the IL-12 polypeptide once about every 2 weeks for at least about 12 weeks. In some aspects, the dose of the IL-12 polypeptide is administered at a therapeutically effective amount of about 12.0 μg of the IL-12 polypeptide once about every 2 weeks for as long as therapeutically effective.

In some aspects, the dose is administered on about days 1 and 15 of a first 28-day cycle. In some aspects, the dose is administered on about days 1 and 15 of a second 28-day cycle. In some aspects, the dose is administered on about day 1 of a third 28-day cycle. In some aspects, the dose is administered on day 1 of each subsequence 28-day cycle. In some aspects, the dose is administered (i) on about days 1 and 15 of a first 28-day cycle and (ii) on about days 1 and 15 of a second 28-day cycle. In some aspects, the dose is administered (i) on about days 1 and 15 of a first 28-day cycle, (ii) on about days 1 and 15 of a second 28-day cycle, and (iii) on about day 1 of a third 28-day cycle. In certain aspects, the dose is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, a dose of about 0.3 μg is administered on about days 1 and 15 of a first 28-day cycle. In some aspects, a dose of about 0.3 μg is administered on about days 1 and 15 of a second 28-day cycle. In some aspects, a dose of about 0.3 μg is administered on about day 1 of a third 28-day cycle. In some aspects, a dose of about 0.3 μg is administered on day 1 of each subsequence 28-day cycle. In some aspects, a dose of about 0.3 μg is administered (i) on about days 1 and 15 of a first 28-day cycle and (ii) on about days 1 and 15 of a second 28-day cycle. In some aspects, a dose of about 0.3 μg is administered (i) on about days 1 and 15 of a first 28-day cycle, (ii) on about days 1 and 15 of a second 28-day cycle, and (iii) on about day 1 of a third 28-day cycle. In certain aspects, a dose of about 0.3 μg is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, a dose of about 1.0 μg is administered on about days 1 and 15 of a first 28-day cycle. In some aspects, a dose of about 1.0 μg is administered on about days 1 and 15 of a second 28-day cycle. In some aspects, a dose of about 1.0 μg is administered on about day 1 of a third 28-day cycle. In some aspects, a dose of about 1.0 μg is administered on day 1 of each subsequence 28-day cycle. In some aspects, a dose of about 1.0 μg is administered (i) on about days 1 and 15 of a first 28-day cycle and (ii) on about days 1 and 15 of a second 28-day cycle. In some aspects, a dose of about 1.0 μg is administered (i) on about days 1 and 15 of a first 28-day cycle, (ii) on about days 1 and 15 of a second 28-day cycle, and (iii) on about day 1 of a third 28-day cycle. In certain aspects, a dose of about 1.0 μg is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, a dose of about 3.0 μg is administered on about days 1 and 15 of a first 28-day cycle. In some aspects, a dose of about 3.0 μg is administered on about days 1 and 15 of a second 28-day cycle. In some aspects, a dose of about 3.0 μg is administered on about day 1 of a third 28-day cycle. In some aspects, a dose of about 3.0 μg is administered on day 1 of each subsequence 28-day cycle. In some aspects, a dose of about 3.0 μg is administered (i) on about days 1 and 15 of a first 28-day cycle and (ii) on about days 1 and 15 of a second 28-day cycle. In some aspects, a dose of about 3.0 μg is administered (i) on about days 1 and 15 of a first 28-day cycle, (ii) on about days 1 and 15 of a second 28-day cycle, and (iii) on about day 1 of a third 28-day cycle. In certain aspects, a dose of about 3.0 μg is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, a dose of about 6.0 μg is administered on about days 1 and 15 of a first 28-day cycle. In some aspects, a dose of about 6.0 μg is administered on about days 1 and 15 of a second 28-day cycle. In some aspects, a dose of about 6.0 μg is administered on about day 1 of a third 28-day cycle. In some aspects, a dose of about 6.0 μg is administered on day 1 of each subsequence 28-day cycle. In some aspects, a dose of about 6.0 μg is administered (i) on about days 1 and 15 of a first 28-day cycle and (ii) on about days 1 and 15 of a second 28-day cycle. In some aspects, a dose of about 6.0 μg is administered (i) on about days 1 and 15 of a first 28-day cycle, (ii) on about days 1 and 15 of a second 28-day cycle, and (iii) on about day 1 of a third 28-day cycle. In certain aspects, a dose of about 6.0 μg is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, a dose of about 12.0 μg is administered on about days 1 and 15 of a first 28-day cycle. In some aspects, a dose of about 12.0 μg is administered on about days 1 and 15 of a second 28-day cycle. In some aspects, a dose of about 12.0 μg is administered on about day 1 of a third 28-day cycle. In some aspects, a dose of about 12.0 μg is administered on day 1 of each subsequence 28-day cycle. In some aspects, a dose of about 12.0 μg is administered (i) on about days 1 and 15 of a first 28-day cycle and (ii) on about days 1 and 15 of a second 28-day cycle. In some aspects, a dose of about 12.0 μg is administered (i) on about days 1 and 15 of a first 28-day cycle, (ii) on about days 1 and 15 of a second 28-day cycle, and (iii) on about day 1 of a third 28-day cycle. In certain aspects, a dose of about 12.0 μg is administered (i) on about days 1 and 15 of a first 28-day cycle, (ii) on about days 1 and 15 of a second 28-day cycle, (iii) on about day 1 of a third 28-day cycle, and (iii) once about every 28 days after the third 28-day cycle.

In some aspects, administration of the IL-12 polypeptide is continued until clinical benefit is achieved.

### II.C. Scaffold-Engineered EVs, e.g., Exosomes

In some aspects, EVs of the present disclosure comprise a membrane modified in its composition. For example, their membrane compositions can be modified by changing the protein, lipid, or glycan content of the membrane.

In some aspects, the surface-engineered EVs are generated by chemical and/or physical methods, such as PEG-induced fusion and/or ultrasonic fusion. In other aspects, the surface-engineered EVs, e.g., exosomes, are generated by genetic engineering. EVs produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface-engineered EVs (e.g., Scaffold X-engineered or Scaffold Y-engineered EVs) can be produced from a cell (e.g., HEK293 cells) transformed with an exogenous sequence encoding a scaffold moiety (e.g., exosome proteins, e.g., Scaffold X and/or Scaffold Y) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EVs that can be purified using the binding agent. Scaffold moieties modified to be more effectively targeted to EVs, e.g., exosomes, and/or membranes can be used. Scaffold moieties modified to comprise a minimal fragment required for specific and effective targeting to EVs, e.g., exosomes, membranes can be also used.

In some aspects, an IL-12 moiety, e.g., polypeptide, disclosed herein is expressed on the surface of an EV, e.g., exosome, as a fusion protein, e.g., fusion protein of an IL-12 moiety, e.g., polypeptide, to a Scaffold X and/or Scaffold Y. For example, the fusion protein can comprise an IL-12 moiety, e.g., polypeptide, disclosed herein linked to a scaffold moiety (e.g., Scaffold X or Scaffold Y).

In certain aspects, Scaffold X comprises the PTGFRN protein, BSG protein, IGSF2 protein, IGSF3 protein, IGSF8 protein, ITGB1 protein, ITGA4 protein, SLC3A2 protein, ATP transporter protein, Lamp-1 protein, Lamp-2 protein, CD13 protein, CD86 protein, Flotillin protein, Syntaxin-3 protein, CD2 protein, CD36 protein, CD40 protein, CD40L protein, CD41a protein, CD44 protein, CD45 protein, ICAM-1 protein, Integrin alpha4 protein, LiCAM protein, LFA-1 protein, Mac-1 alpha and beta protein, Vti-iA and B protein, CD3 epsilon and zeta protein, CD9 protein, CD18 protein, CD37 protein, CD53 protein, CD63 protein, CD81 protein, CD82 protein, CXCR4 protein, FcR protein, GluR2/3 protein, HLA-DM (MHIC II) protein, immunoglobulins protein, MHIC-I or MHC-II components protein, TCR beta protein, tetraspanin protein, or a fragment or a variant thereof.

In some aspects, the surface-engineered EVs, e.g., exosomes (e.g., Scaffold X-engineered EVs, e.g., exosomes) described herein demonstrate superior characteristics compared to EVs, e.g., exosomes, known in the art. For example, surface (e.g., Scaffold X)-engineered contain modified proteins more highly enriched on their surface than naturally occurring EVs, e.g., exosomes, or the EVs, e.g., exosomes, produced using conventional exosome proteins. Moreover, the surface-engineered EVs, e.g., exosomes, (e.g., Scaffold X-engineered EVs, e.g., exosomes) of the present invention can have greater, more specific, or more controlled biological activity compared to naturally occurring EVs, e.g., exosomes, or the EVs, e.g., exosomes, produced using conventional exosome proteins.

In other aspects, the EVs, e.g., exosomes, of the present disclosure contains an IL-12 moiety, e.g., polypeptide, and a Scaffold X, wherein the IL-12 moiety is linked to the Scaffold X.

In some aspects, Scaffold X useful for the present disclosure comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 2 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33.

TABLE 2A
Exemplary Scaffold X Protein Sequences

| Protein | Sequence |
|---|---|
| The PTGFRN | MGRLASRPLLLALLSLALCRGRVVRVPTATLVRVVGTELVIPCNVSDYDGPSEQNFDWSFSSLGSS |
| Protein | FVELASTWEVGFPAQLYQERLQRGEILLRRTANDAVELHIKNVQPSDQGHYKCSTPSTDATVQGNY |
| (SEQ ID NO: | EDTVQVKVLADSLHVGPSARPPPSLSLREGEPFELRCTAASASPLHTHLALLWEVHRGPARRSVLA |
| 1) | LTHEGRFHPGLGYEQRYHSGDVRLDTVGSDAYRLSVSRALSADQGSYRCIVSEWIAEQGNWQEIQE |
| | KAVEVATVVIQPSVLRAAVPKNVSVAEGKELDLTCNITTDRADDVRPEVTWSFSRMPDSTLPGSRV |
| | LARLDRDSLVHSSPHVALSHVDARSYHLLVRDVSKENSGYYYCHVSLWAPGHNRSWHKVAEAVSSP |
| | AGVGVTWLEPDYQVYLNASKVPGFADDPTELACRVVDTKSGEANVRFTVSWYYRMNRRSDNVVTSE |
| | LLAVMDGDWTLKYGERSKQRAQDGDFIFSKEHTDTFNFRIQRTTEEDRGNYYCVVSAWTKQRNNSW |
| | VKSKDVFSKPVNIFWALEDSVLVVKARQPKPFFAAGNTFEMTCKVSSKNIKSPRYSVLIMAEKPVG |
| | DLSSPNETKYIISLDQDSVVKLENWTDASRVDGVVLEKVQEDEFRYRMYQTQVSDAGLYRCMVTAW |
| | SPVRGSLWREAATSLSNPIEIDFQTSGPIFNASVHSDTPSVIRGDLIKLFCIITVEGAALDPDDMA |
| | FDVSWFAVHSFGLDKAPVLLSSLDRKGIVTTSRRDWKSDLSLERVSVLEFLLQVHGSEDQDFGNYY |
| | CSVTPWVKSPTGSWQKEAEIHSKPVFITVKMDVLNAFKYPLLIGVGLSTVIGLLSCLIGYCSSHWC |
| | CKKEVQETRRERRRLMSMEMD |
| The PTGFRN | GPIFNASVHSDTPSVIRGDLIKLFCIITVEGAALDPDDMAFDVSWFAVHSFGLDKAPVLLSSLDRK |
| protein | GIVTTSRRDWKSDLSLERVSVLEFLLQVHGSEDQDFGNYYCSVTPWVKSPTGSWQKEAEIHSKPVF |
| Fragment | ITVKMDVLNAFKYPLLIGVGLSTVIGLLSCLIGYCSSHWCCKKEVQETRRERRRLMSMEM |
| (SEQ ID NO: | 687-878 of SEQ ID NO: 1 |
| 33) | |

In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 2, 3, 4, 5, 6, 7, or 8.

Non-limiting examples of other Scaffold X proteins that can be used to link an IL-12 moiety, e.g., polypeptide, to the surface of EVs, e.g., exosomes, can be found at U.S. Pat. Nos. 10,195,290 B1 and 10,561,740 B2, each of which is incorporated by reference in its entirety.

In some aspects, Scaffold X described herein can also be used to link an IL-12 moiety on the luminal surface and/or on the exterior surface of the EVs, e.g., exosomes, at the same time. For example, the PTGFRN polypeptide can be used to link an IL-12 moiety inside the lumen in addition to the surface of the EV, e.g., exosome. In some aspects, a Scaffold X can be used to link an IL-12 moiety and an additional therapeutic agent to the EVs, e.g., exosomes, (e.g., payload). Therefore, in certain aspects, Scaffold X disclosed herein can be used for dual purposes.

In some aspects, EVs, e.g., exosomes, of the present disclosure comprise an internal space (i.e., lumen) that is different from that of the naturally occurring EVs, e.g., exosomes. For example, the EV, e.g., exosome, can be changed such that the composition in the luminal side of the EV, e.g., exosome, has the protein, lipid, or glycan content different from that of the naturally-occurring EVs, e.g., exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal side of the EV, e.g., exosome. Various modifications or fragments of the exosome protein that can be expressed in the luminal side of the EV, e.g., exosome, can be used for the aspects of the present disclosure.

In some aspects, an IL-12 moiety disclosed herein is in the lumen of the EV, e.g., exosome (i.e., encapsulated). In some aspects, an IL-12 moiety is linked to the luminal surface of the EV, e.g., exosome. As used herein, when a molecule (e.g., an IL-12 moiety) is described as “in the lumen” of the EV, e.g., exosome, it means that the molecule is located within the EV, e.g., exosome (e.g., associated), but is not linked to any molecule on the luminal surface of EVs. In other aspects, an IL-12 moiety is expressed on the luminal surface of the EV, e.g., exosome, as a fusion molecule, e.g., fusion molecule of an IL-12 moiety to a scaffold moiety (e.g., Scaffold X or Scaffold Y).

### II.D. Linkers

The EVs of the present disclosure can comprises one or more linkers that link the IL-12 moiety to EVs or to a scaffold moiety, e.g., Scaffold X on the exterior surface of the EVs. In some aspects, the IL-12 moiety is linked to the EVs directly or in a scaffold moiety on the EVs by a linker. In some aspects, the IL-12 is linked to a lipid bilayer of the EV, e.g., by a linker. The linker can be any chemical moiety known in the art.

In some aspects, the term “linker” refers to a peptide or polypeptide sequence (e.g., a synthetic peptide or polypeptide sequence) or to a non-polypeptide. In some aspects, two or more linkers can be linked in tandem. Generally, linkers provide flexibility or prevent/ameliorate steric hindrances. Linkers are not typically cleaved; however in certain aspects, such cleavage can be desirable. Accordingly, in some aspects a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

Linkers may be susceptible to cleavage (“cleavable linker”) thereby facilitating release of the IL-12 moiety or other payloads. In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof. In some aspects, the linker comprises a non-cleavable liker.

### III.E. Producer Cells and Modifications

EVs, e.g., exosomes, can be produced from a cell grown in vitro or a body fluid of a subject. When EVs, e.g., exosomes, are produced from in vitro cell culture, various producer cells, e.g., HEK293 cells, can be used. Additional cell types that can be used for the production of the lumen-engineered EVs, e.g., exosomes, described herein include, without limitation, mesenchymal stem cells, T-cells, B-cells, dendritic cells, macrophages, and cancer cell lines. Further examples include: Chinese hamster ovary (CHO) cells, mesenchymal stem cells (MSCs), BJ human foreskin fibroblast cells, fHDF fibroblast cells, AGE.HN© neuronal precursor cells, CAP© amniocyte cells, adipose mesenchymal stem cells, and RPTEC/TERT1 cells. In certain aspects, a producer cell is not a dendritic cell, macrophage, B cell, mast cell, neutrophil, Kupffer-Browicz cell, cell derived from any of these cells, or any combination thereof.

Some aspects may also include genetically modifying the EV, e.g., exosome, to comprise one or more exogenous sequences, e.g., PTGFRN linked to IL-12 to produce modified EVs that express exogenous proteins on the vesicle surface.

More specifically, the EV, e.g., exosome, of the present can be produced from a cell transformed with a sequence encoding one or more additional exogenous proteins including, but not limited to ligands, cytokines, or antibodies, or any combination thereof. These additional exogenous proteins may enable activation or modulation of additional immune stimulatory signals. Exemplary additional exogenous proteins contemplated for use include the proteins, ligands, and other molecules described in detail in U.S. Patent Application 62/611,140, International Publication No. WO/2019/133934, and U.S. Pat. Nos. 10,195,290 B1 and 10,561,740 B2, each of which is incorporated herein by reference in its entirety. In some aspects, the EV, e.g., exosome, is further modified with a ligand comprising CD40L, OX40L, or CD27L. In some aspects, the EV, e.g., exosome, is further modified with a cytokine comprising IL-7 or IL-15. Any of the one or more exosome proteins described herein can be expressed from a plasmid, an exogenous sequence inserted into the genome or other exogenous nucleic acid such as a synthetic messenger RNA (mRNA).

### III. EV Purification

The EVs, e.g., exosomes, prepared for the present disclosure can be isolated from the producer cells. It is contemplated that all known manners of isolation of EVs, e.g., exosomes, are deemed suitable for use herein. For example, physical properties of EVs, e.g., exosomes, may be employed to separate them from a medium or other source material, including separation on the basis of electrical charge (e.g., electrophoretic separation), size (e.g., filtration, molecular sieving, etc), density (e.g., regular or gradient centrifugation), Svedberg constant (e.g., sedimentation with or without external force, etc). Alternatively, or additionally, isolation may be based on one or more biological properties, and include methods that may employ surface markers (e.g., for precipitation, reversible binding to solid phase, FACS separation, specific ligand binding, non-specific ligand binding, etc.). In yet further contemplated methods, the EVs, e.g., exosomes, may also be fused using chemical and/or physical methods, including PEG-induced fusion and/or ultrasonic fusion.

The EVs, e.g., exosomes, may also be purified after incubation with the STING agonist to remove free, unencapsulated STING agonist from the composition. All manners of previously disclosed methods are also deemed suitable for use herein, including separation on the basis of physical or biological properties of EVs, e.g., exosomes.

Isolation, purification, and enrichment can be done in a general and non-selective manner (typically including serial centrifugation). Alternatively, isolation, purification, and enrichment can be done in a more specific and selective manner (e.g., using producer cell-specific surface markers). For example, specific surface markers may be used in immunoprecipitation, FACS sorting, affinity purification, bead-bound ligands for magnetic separation etc.

In some aspects, size exclusion chromatography can be utilized to isolate or purify the EVs, e.g., exosomes. Size exclusion chromatography techniques are known in the art. Exemplary, non-limiting techniques are provided herein. In some aspects, a void volume fraction is isolated and comprises the EVs, e.g., exosomes, of interest. In some aspects, for example, density gradient centrifugation can be utilized to further isolate the EVs, e.g., exosomes. Still further, in some aspects, it can be desirable to further separate the producer cell-derived EVs, e.g., exosomes, from EVs of other origin. For example, the producer cell-derived EVs, e.g., exosomes, can be separated from non-producer cell-derived EVs, e.g., exosomes, by immunosorbent capture using an antigen antibody specific for the producer cell.

In some aspects, the isolation of EVs, e.g., exosomes, may involve size exclusion chromatography or ion chromatography, such as anion exchange, cation exchange, or mixed mode chromatography. In some aspects, the isolation of EVs, e.g., exosomes, may involve desalting, dialysis, tangential flow filtration, ultrafiltration, or diafiltration, or any combination thereof0. In some aspects, the isolation of EVs, e.g., exosomes, may involve combinations of methods that include, but are not limited to, differential centrifugation, size-based membrane filtration, concentration and/or rate zonal centrifugation. In some aspects, the isolation of EVs, e.g., exosomes, may involve one or more centrifugation steps. The centrifugation may be performed at about 50,000 to 150,000×g. The centrifugation may be performed at about 50,000×g, 75,000×g, 100,000×g, 125,000×g, or 150,000×g.

### IV. Therapeutic Administration

### IV.A. Methods of Treating Cancer

Provided herein are methods of treating cancer in a subject. The method comprises administering to the subject a dose of an IL-12 polypeptide, wherein the amount of the IL-12 polypeptide in the dose is about 0.1 μg to about 30 μg. In some aspects, the IL-12 moiety, e.g., polypeptide, is administered intra-tumorally to the subject. In some aspects, the IL-12 moiety, e.g., polypeptide, is administered parenterally, orally, intravenously, intramuscularly, intraperitoneally, or via any other appropriate administration route.

Also provided herein are methods of preventing metastasis of cancer in a subject. The method comprises administering to the subject a dose of an IL-12 polypeptide, wherein the amount of the IL-12 polypeptide in the dose is about 0.1 μg to about 30 μg. In some aspects, the IL-12 moiety, e.g., polypeptide, is administered intratumorally in a first tumor in one location, and the IL-12 moiety, e.g., polypeptide, administered in a first tumor prevents metastasis of one or more tumors at a second location.

In some aspects, administering an IL-12 moiety, e.g., polypeptide, disclosed herein inhibits and/or reduces tumor growth in a subject. In some aspects, the tumor growth (e.g., tumor volume or weight) is reduced by at least about 5%, at least about 10%, at least about 20%, at least about 30%, at least about 40%, at least about 50%, at least about 60%, at least about 70%, at least about 80%, at least about 90%, or about 100% compared to a reference (e.g., tumor volume in a corresponding subject after administration an EV, e.g., exosome, without the IL-12 moiety).

In some aspects, the cancer being treated is characterized by infiltration of leukocytes (T-cells, B-cells, macrophages, dendritic cells, monocytes) into the tumor microenvironment, or so-called “hot tumors” or “inflammatory tumors”. In some aspects, the cancer being treated is characterized by low levels or undetectable levels of leukocyte infiltration into the tumor microenvironment, or so-called “cold tumors” or “non-inflammatory tumors”. In some aspects, an EV, e.g., exosome, is administered in an amount and for a time sufficient to convert a “cold tumor” into a “hot tumor”, i.e., said administering results in the infiltration of leukocytes (such as T-cells) into the tumor microenvironment. In certain aspects, cancer comprises bladder cancer, cervical cancer, renal cell cancer, testicular cancer, colorectal cancer, lung cancer, head and neck cancer, and ovarian, lymphoma, liver cancer, glioblastoma, melanoma, myeloma, leukemia, pancreatic cancers, or combinations thereof. The term “distal tumor”, “distant tumor”, or “secondary tumor” as used herein refers to a tumor that has spread from the original (or primary) tumor to distant organs or distant tissues, e.g., lymph nodes. In some aspects, the EVs, e.g., exosomes, of the disclosure treats a tumor after the metastatic spread.

Non-limiting examples of cancers (or tumors) that can be treated with methods disclosed herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer (e.g., choriocarcinoma and non-seminoma), carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated ANIL (MO), myeloblastic leukemia (M1), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukaemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTCL) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, peritoneal cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In certain aspects, a cancer that can be treated with the present disclosure is a pancreatic cancer and/or a peritoneal cancer. In some aspects, a cancer (or tumor) that can be treated comprises a bladder cancer. In some aspects, the composition is delivered to a subject by intravesical administration, wherein the subject has a bladder cancer.

In some aspects, the methods described herein can also be used for treatment of metastatic cancers, unresectable, refractory cancers (e.g., cancers refractory to previous cancer therapy), and/or recurrent cancers.

In some aspects, EVs, e.g., exosomes, disclosed herein can be used in combination with one or more additional anti-cancer and/or immunomodulating agents. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy).

Some aspects of the present disclosure are directed to methods of administering to a subject in need thereof (i) a dose of an IL-12 polypeptide, wherein the amount of the IL-12 polypeptide in the dose is about 0.1 μg to about 30 μg; and (ii) an additional therapy. In some aspects, the additional therapy is an additional anticancer agent and/or immunomodulating agent. Such agents can include, for example, chemotherapy drugs, small molecule drugs, or antibodies that stimulate the immune response to a given cancer. In some aspects, the methods described herein are used in combination with a standard of care treatment (e.g., surgery, radiation, and chemotherapy).

In some aspects, the IL-12 moiety, e.g., associated with an EV (e.g., exosome), disclosed herein can be used in combination with one or more additional therapeutic agents (e.g., immuno-oncology agents), such that multiple elements of the immune pathway can be targeted. Non-limiting of such combinations include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxygenase (IDO), dioxygenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, the additional anticancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, an immuno-oncology agent comprises an immune checkpoint activator (i.e., promotes signaling through the particular immune checkpoint pathway). In certain aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, the additional anticancer agent comprises a standard of care chemotherapy. In some aspects, the standard of care chemotherapy comprises a platinum-based chemotherapy. In some aspects, the standard of care chemotherapy comprises a platinum-based doublet chemotherapy.

In some aspects, the additional anticancer agent is a standard of care therapy for treating colorectal cancer. In some aspects, the additional anticancer agent comprises 5-fluorouracil (FU), leucovorin (LV), oxaliplatin, irinotecan, an anti-vascular endothelial growth factor (VEGF) antibody, an anti-epidermal growth factor receptor (EGFR) antibody, or any combination thereof. In some aspects, the additional anticancer agent comprises FU, LV, and oxaliplatin (FOLFOX). In some aspects, the additional anticancer agent comprises FU, LV, and irinotecan (FOLFIRI). In some aspects, the subject has MSI-high colorectal cancer. In some aspects, the subject has colorectal cancer presenting with one or more KRAS mutations, e.g., at position Gly12, Gly13, Glu61, or any combination thereof. In some aspects, the subject has colorectal cancer presenting with one or more BRAF mutations, e.g., BRAF V600E.

In some aspects, the additional anticancer agent is a standard of care therapy for treating HCC. In some aspects, the additional anticancer agent comprises sorafenib. In some aspects, the additional anticancer agent comprises lenvatinib. In some aspects, the additional anticancer agent comprises atezolizumab and bevacizumab. In some aspects, the additional anticancer agent comprises pembrolizumab, nivolumab, ipilimumab, or a combination thereof. In some aspects, the additional anticancer agent comprises FU, LV, and oxaliplatin (FOLFOX). In some aspects, the additional anticancer agent comprises regorafenib, cabozantinib, ramucirumab, or any combination thereof.

In some aspects, the additional anticancer agent is a standard of care therapy for treating gastric cancer. In some aspects, the additional anticancer agent comprises (i) epirubicin, cisplatin, and 5-FU (“ECF”)′ (ii) or epirubicin, cisplatin, and capecitabine (“ECX”); or (iii) docetaxel, oxaliplatin, and 5-FU/leucovorin (“FLOT”). In some aspects, the additional anticancer agent comprises a HER2-targeting agent.

In some aspects, a combination of an IL-12 moiety, e.g., associated with an EV, e.g., exosome, disclosed herein and a second agent discussed herein (e.g., immune checkpoint inhibitor) can be administered concurrently as a single composition in a pharmaceutically acceptable carrier. In other aspects, a combination of an IL-12 moiety, e.g., associated with an EV, e.g., exosome, and a second agent discussed herein (e.g., immune checkpoint inhibitor) can be administered concurrently as separate compositions. In further aspects, a combination of an IL-12 moiety, e.g., associated with an EV, e.g., exosome, and a second agent discussed herein (e.g., immune checkpoint inhibitor) can be administered sequentially. In some aspects, an IL-12 moiety, e.g., associated with an EV, e.g., exosome, is administered prior to the administration of a second agent (e.g., immune checkpoint inhibitor).

### IV.A.1. Cutaneous T-Cell Lymphoma

In some aspects of the present disclosure, the cancer (tumor) treatable by the methods disclosed herein comprises cutaneous T-cell lymphoma (CTCL). As used herein, CTCL refers to a type of non-Hodgkin T-cell lymphoma that affects the skin. There are several subtypes of CTCL, including but not limited to mycosis fungoides (MF), Sezary syndrome (SS), and a spectrum of T-cell neoplasms referred to as CD30-positive lymphoproliferative disorders. As such, in certain aspects, the methods disclosed herein treat an MF in a subject in need thereof. In some aspects, the methods disclosed herein treat an SS in a subject in need thereof. In some aspects, the methods disclosed herein treat a CD30-positive lymphoproliferative disorder.

In some aspects, the CTCL comprises an aggressive phenotype. In some aspects, the CTCL comprises an indolent, i.e., slow growing, phenotype.

There are four main stages of CTCL, e.g., MF, SS, and/or a CD30-positive lymphoproliferative disorder. Stage 1 CTCL is characterized by a phenotype that only affects the skin, presenting as patches or plaques. During stage 1A, less than one tenth of the skin is affected; and during stage 1B, one tenth or more of the skin is affected. Stage 2 CTCL is characterized by patches or plaques on the skin and enlarged lymph nodes, which lack abnormal lymphoma cells (stage 2A); or presentation of one or more raised lumps or tumors on the skin, with or without enlarged lymph nodes (stage 2B). Stage 3 CTCL is characterized by patches or plaques affecting four-fifths or more of the skin, with general redness, swelling, itching, and sometimes pain (erythroderma) of the skin; enlarged lymph nodes, which lack abnormal lymphoma cells; and either few or no lymphoma cells in the bloodstream (erythrodermic mycosis fungoides; Stage 3A) or moderate numbers of lymphoma cells in the bloodstream (Sezary syndrome; Stage 3B). Stage 4 CTCL affects the skin and has also spread to the bloodstream, lymph nodes, or other organs. Stage 4A refers to presentation of numerous abnormal lymphoma cells in the bloodstream (Sezary syndrome) or lymphoma cells in the lymph nodes; and Stage 4B refers to the spread of the lymphoma to other organs.

In some aspects, the methods disclosed herein treat a stage 1A CTCL. In some aspects, the methods disclosed herein treat a stage 1B CTCL. In some aspects, the methods disclosed herein treat a stage 2A CTCL. In some aspects, the methods disclosed herein treat a stage 2B CTCL. In some aspects, the methods disclosed herein treat a stage 3A CTCL. In some aspects, the methods disclosed herein treat a stage 3B CTCL. In some aspects, the methods disclosed herein treat a stage 4A CTCL. In some aspects, the methods disclosed herein treat a stage 4B CTCL.

### IV.A.2. Merkel Cell Carcinoma

In some aspects of the present disclosure, the cancer (tumor) treatable by the methods disclosed herein comprises Merkel cell carcinoma (MCC). As used herein, MCC, or neuroendocrine carcinoma of the skin, refers to a rare type of skin cancer characterized by a flesh-colored or bluish-red nodule, often presenting on the face, head or neck. MCC is generally an aggressive form of skin cancer that has a high risk for recurrence and metastasis.

Standard of care therapy for MCC includes radiotherapy, surgery, systemic therapies or chemotherapy, or a combination thereof. In addition, antibodies that inhibit the interaction of programmed death 1 (PD-1) and its ligand (PD-L1) have been approved for the treatment of MCC, including avelumab.

Stage 0 MCC refers to presentation of abnormal Merkel cells in the top layer of the skin. Stage 1 MCC refers to presentation of a tumor that is 2 cm or smaller in diameter. Stage 2 MCC refers to either a tumor that is larger than 2 cm (stage 2A) or to a tumor that has spread to nearby connective tissue, muscle, cartilage, or bone (stage 2B). Stage 3A MCC refers to (i) a tumor of any size that has spread to nearby connective tissue, muscle, cartilage, or bone, and wherein cancer is found in the lymph node; or (ii) spread to lymph node can be observed by physical exam and confirmed by biopsy. Stage 3B MCC refers to presentation of a tumor of any size, which has (i) spread to nearby connective tissue, muscle, cartilage, or bone and wherein cancer is found in the lymph node; or (ii) spread to a lymph vessel between the primary tumor and proximal and/or distal lymph nodes. Stage IV MCC refers to metastasis of the cancer to a distal location on the skin or to other organs, e.g., liver, lung, bone, or brain.

In some aspects, the methods disclosed herein treat a stage 0 MCC. In some aspects, the methods disclosed herein treat a stage 1 MCC. In some aspects, the methods disclosed herein treat a stage 2A MCC. In some aspects, the methods disclosed herein treat a stage 2B MCC. In some aspects, the methods disclosed herein treat a stage 3A MCC. In some aspects, the methods disclosed herein treat a stage 3B MCC. In some aspects, the methods disclosed herein treat a stage 4 MCC. In some aspects, the methods disclosed herein treat recurrent MCC.

### IV.A.3. Kaposi Sarcoma

In some aspects of the present disclosure, the cancer (tumor) treatable by the methods disclosed herein comprises Kaposi sarcoma. As used herein, Kaposi sarcoma refers to a cancer that develops from the cells that line lymph or blood vessels, which usually appears as tumors (purple, red, or brown blotches) on the skin, on mucosal surfaces such as inside the mouth, or less frequently in other parts of the body including in the lymph nodes.

There are at least four types of Kaposi sarcomas: epidemic Kaposi sarcoma (which develops in people infected with HIV), classic Kaposi sarcoma, endemic Kaposi sarcoma, and iatrogenic (transplant-related) Kaposi sarcoma. Often, Kaposi sarcoma is associated with a weakened immune system, e.g., in malnourished or elderly patients or patients suffering from a chronic viral infection (e.g., HIV or HSV).

Kaposi sarcoma is typically treated by immune reconstitution, in particular for HIV patients. Chemotherapy can also be administered, including but not limited to doxorubicin, daunorubicine, paclitaxel, and combinations thereof.

### IV.A.4. Triple-Negative Breast Cancer

In some aspects of the present disclosure, the cancer (tumor) treatable by the methods disclosed herein comprises triple-negative breast cancer (TNBC). As used herein, TNBC refers to a breast cancer characterized by tumors that test negative for expression of estrogen receptor (ER-negative), progesterone receptor (PR-negative), and HER2 (HER2-negative). TNBC is considered an aggressive cancer, which grows quickly, which is more likely to have spread at the time of diagnosis, and which a higher incidence of relapse after therapy than other forms of breast cancer. As a result, TNBC has a lower survival rate than other breast cancers, with the 5-year survival rate for localized TNBC being about 91%, regional TNBC being about 65%, and distant TNBC being about 11%.

As TNBC lacks expression of ER, PR, and HER2, there are limited treatments available for TNBC. For non-metastatic TNBC, chemotherapy followed by surgery is the primary treatment option. Metastatic TNBC is typically treated using chemotherapy (e.g., PARP inhibitors, platinum-based chemotherapies, and/or immunotherapies).

Stage 0 TNBC refers to non-invasive breast cancers, such as DCIS (ductal carcinoma in situ). In stage 0, there is no evidence of cancer cells or non-cancerous abnormal cells breaking out of the part of the breast in which they started, or getting through to or invading neighboring normal tissue. Stage I TNBC refers to invasive breast cancer (cancer cells are observed breaking through to or invading normal surrounding breast tissue). Stage 1A refers to invasive breast cancer in which the tumor measures up to 2 centimeters (cm) and the cancer has not spread outside the breast (e.g., the lymph nodes are not affected). Stage 1B refers to invasive breast cancer in which (i) small groups of cancer cells (0.2 mm to 2 mm in diameter) are found in the lymph nodes but there is no tumor presenting in the breast, or (ii) there is a tumor presenting in the breast that is no larger than 2 cm, and there are small groups of cancer cells (0.2 mm to 2 mm in diameter) in the lymph nodes. Microscopic invasion is possible in stage 1 breast cancer. In microscopic invasion, the cancer cells have just started to invade the tissue outside the lining of the duct or lobule, but the invading cancer cells do not measure more than 1 mm. For HER2-negative breast cancers (e.g., TNBC), a tumor that is larger than 2 cm but not larger than 5 cm and that has not spread to the lymph nodes is considered as stage 1. Stage 2A refers to a breast cancer wherein (i) no tumor can be found in the breast, but cancer (larger than 2 mm) is found in one to three axillary lymph nodes or in the lymph nodes near the breast bone; or (ii) the tumor measures 2 cm or smaller and has spread to the axillary lymph nodes. Stage 2B refers to invasive breast cancers in which (i) the tumor is 2 cm to 5 cm in size, with presentation of small groups of breast cancer cells (0.2 to 2 mm in size) in the lymph nodes; (ii) the tumor is 2 cm to 5 cm in size; with metastasis to one to three axillary lymph nodes or to lymph nodes near the breastbone; or (iii) the tumor is larger than 5 cm but has not spread to the axillary lymph nodes. Stage 3A refers to invasive breast cancer in which (i) no tumor is found in the breast or the tumor may be any size, and there is metastasis to four to nine axillary lymph nodes or in the lymph nodes near the breastbone; (ii) the tumor is larger than 5 cm and small groups of breast cancer cells (0.2 mm to 2 mm in size) are found in the lymph nodes; or (iii) the tumor is larger than 5 cm and the cancer has spread to one to three axillary lymph nodes or to the lymph nodes near the breastbone. Stage 3B describes invasive breast cancer in which the tumor is any size and has spread to the chest wall and/or skin of the breast, causing swelling or an ulcer and the tumor has spread (i) to up to nine axillary lymph nodes or (ii) to lymph nodes near the breastbone. Stage 3C refers to invasive breast cancer in which there may be no sign of cancer in the breast or, if there is a tumor, it may be any size and may have spread to the chest wall and/or the skin of the breast and the cancer has spread (i) to ten or more axillary lymph nodes, (ii) to lymph nodes above or below the collarbone, or (iii) to lymph nodes near the breastbone. Stage 4, or advanced, breast cancer refers to invasive breast cancer that has spread beyond the breast and nearby lymph nodes to other organs of the body, such as the lungs, distant lymph nodes, skin, bones, liver, or brain.

In some aspects, the methods disclosed herein treat a stage 0 TNBC. In some aspects, the methods disclosed herein treat a stage 1A TNBC. In some aspects, the methods disclosed herein treat a stage 1B TNBC. In some aspects, the methods disclosed herein treat a stage 2A TNBC. In some aspects, the methods disclosed herein treat a stage 2B TNBC. In some aspects, the methods disclosed herein treat a stage 3A TNBC. In some aspects, the methods disclosed herein treat a stage 3B TNBC. In some aspects, the methods disclosed herein treat a stage 3C TNBC. In some aspects, the methods disclosed herein treat a stage 4 TNBC. In some aspects, the methods disclosed herein treat recurrent TNBC.

### IV.A.5. Glioblastoma

In some aspects of the present disclosure, the cancer (tumor) treatable by the methods disclosed herein comprises a glioblastoma. As used herein, glioblastoma, also referred to as “glioblastoma multiforme” or “GBM,” refers to a type of cancer that is a subset of Stage IV astrocytoma. There are several variants of glioblastoma, including giant cell glioblastoma, gliosarcoma, classical glioblastoma, neural glioblastoma, proneural glioblastoma, and mesenchymal glioblastoma. Glioblastoma tumors can be primary (de novo) or secondary.

There is currently no cure for glioblastoma. Standard of care treatment for glioblastoma is surgical resection followed by radiation and chemotherapy (e.g., with temozolomide). Bevacizumab can also be used as a second-line therapy for glioblastoma. The median length of survival after a diagnosis is 15-18 months, while the disease's five-year survival rate is around 10%. Though all glioblastomas recur, initial treatments may keep the tumor controlled for months or even years.

### EXAMPLES

### Example 1: Administration of Exosomes Comprising Surface-Displayed IL-12 in a Mouse Cancer Model

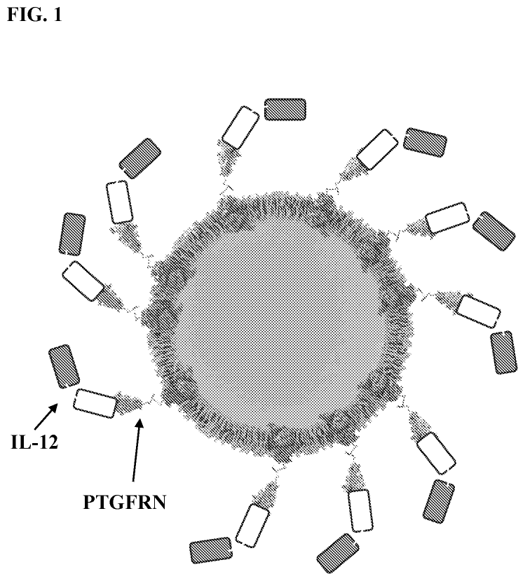

Exosomes with surface-displayed IL-12 were prepared by expressing in exosome-producing cells a fusion construct comprising a single peptide IL-12 (p35 linked by a peptide linker p40) linked to PTGFRN (FIG. 1). Potency was assessed in vitro using human PBMCs or murine splenocytes and in vivo using mouse subcutaneous tumor models. Local versus systemic pharmacology was determined with intratumoral injection in mice and subcutaneous injection in monkeys. All studies were benchmarked against recombinant IL-12 (rIL-12).

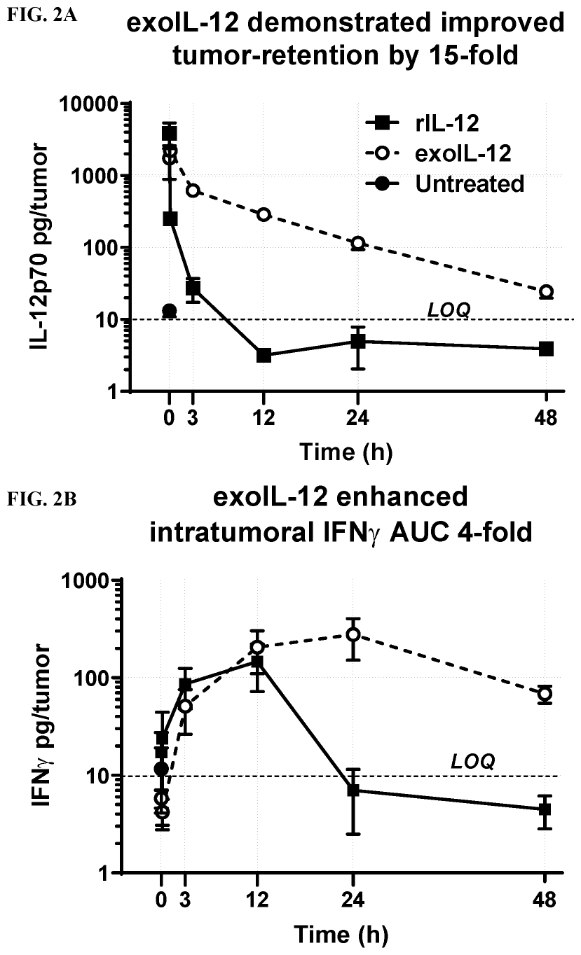

In an MC38 mouse tumor model, intratumoral administration of exosomes with surface-displayed IL-12 (exoIL-12) showed enhanced PK and sustained PD as compared to free recombinant IL-12 and untreated controls. Mice administered exoIL-12 showed increased tumor retention (of about 15-fold) as measured by IL-12p70 concentration per tumor at 3, 12, 24, and 48 hours post administration, compared with free recombinant IL-12 (FIG. 2A). In addition, exoIL-12 administration led to enhanced intratumoral IFN-gamma AUC by about 4-fold as compared to recombinant IL-12 (FIG. 2B). Further, intratumoral administration of exoIL-12 led to a dose dependent reduction in MC38 tumor growth in mice. ExoIL-12 was 100-fold more potent than rIL-12 in tumor growth inhibition, with mice receiving 100 ng exoIL-12 showing little to no tumor growth (FIG. 2C). In the MC38 tumor model, complete responses were observed in 63% of mice treated with exoIL-12; in contrast, rIL-12 resulted in 0% complete responses at an equivalent IL-12 dose. This correlated with dose-dependent increases in tumor antigen-specific CD8+ T cells, which increased nearly 4-fold in exoIL-12 treated mice (FIG. 2E).

Suppression of tumor growth was again observed following MC38 rechallenge (FIG. 2D). Re-challenge studies of exoIL-12 complete responder mice showed no tumor regrowth and depletion of CD8+ T cells completely abrogated antitumor activity of exoIL-12. Following intratumoral administration, exoIL-12 exhibited 10-fold higher intratumoral exposure than rIL-12 and prolonged IFNγ production up to 48 hr. Retained local pharmacology of exoIL-12 was further confirmed using subcutaneous injections in non-human primates.

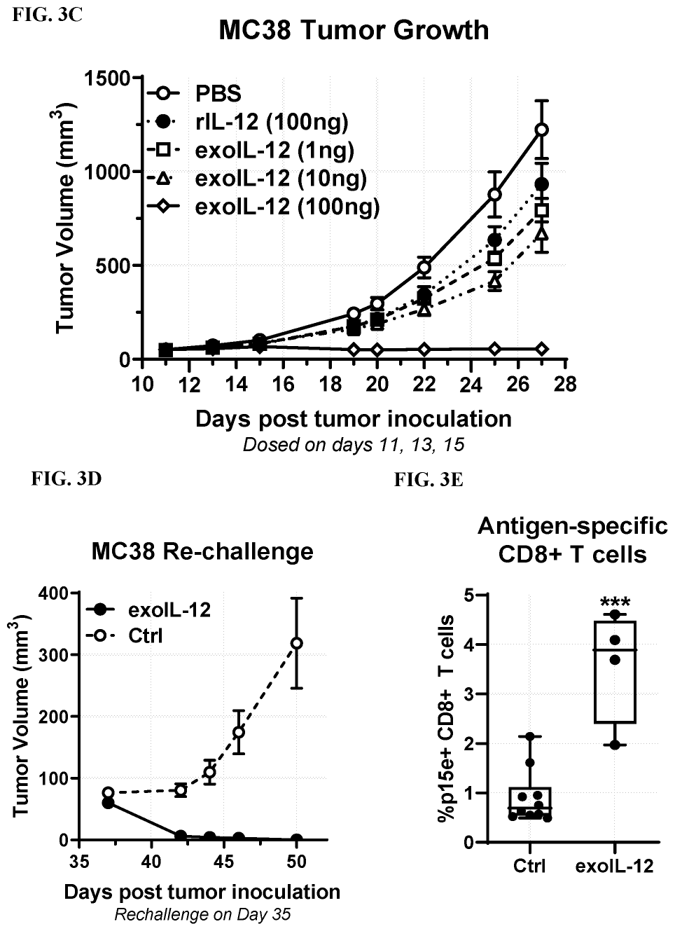

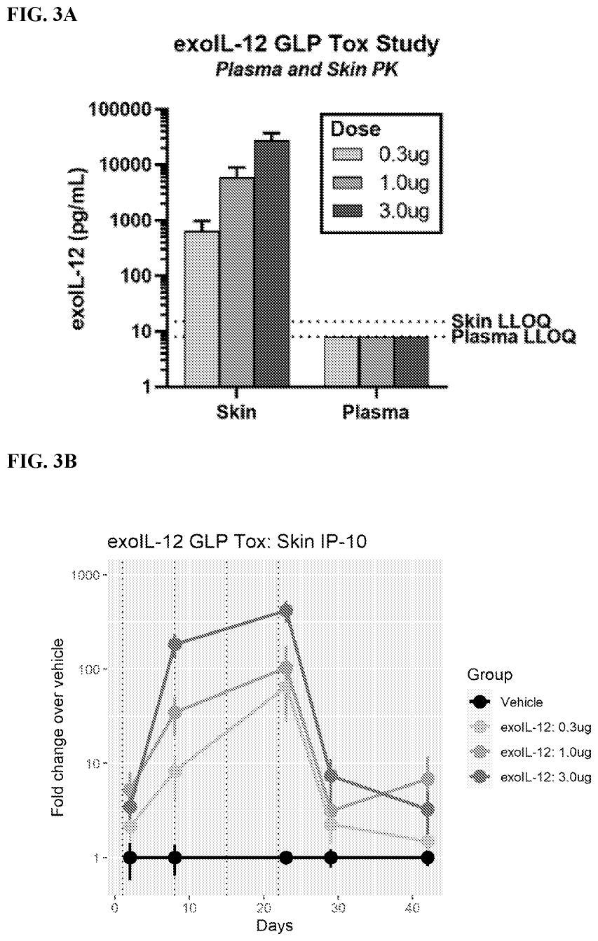

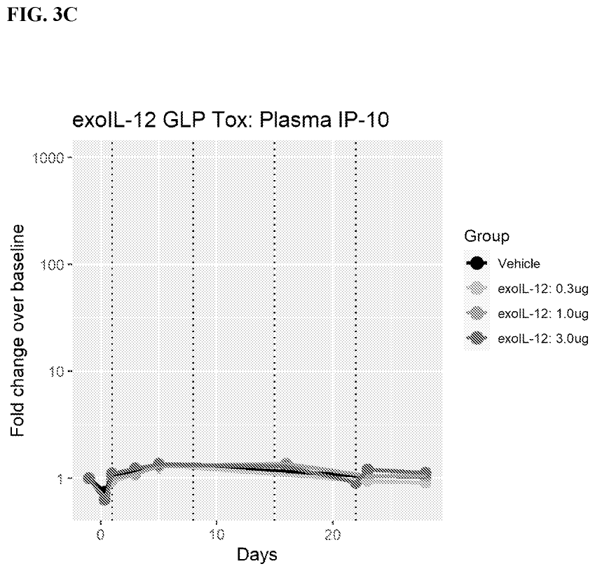

Toxicology analysis revealed that the highest dose tested, 3 μg exoIL-12, was NOAEL (no observable adverse effect level), showing limited plasma levels and dose dependent tissue levels (FIG. 3A). CXCL10/IP-10 expression was observed to be sustained in the skin after a single dose but was undetectable in plasma (FIGS. 3B-3C).

Tumor-restricted pharmacology of exoIL-12 results in superior in vivo efficacy and immune memory without systemic IL-12 exposure and related toxicity. As such, exoIL-12 overcomes key limitations of rIL-12.

### Example 2: Clinical Trial Studying the Safety and Efficacy of Administration of Exosomes Comprising Surface-Displayed IL-12

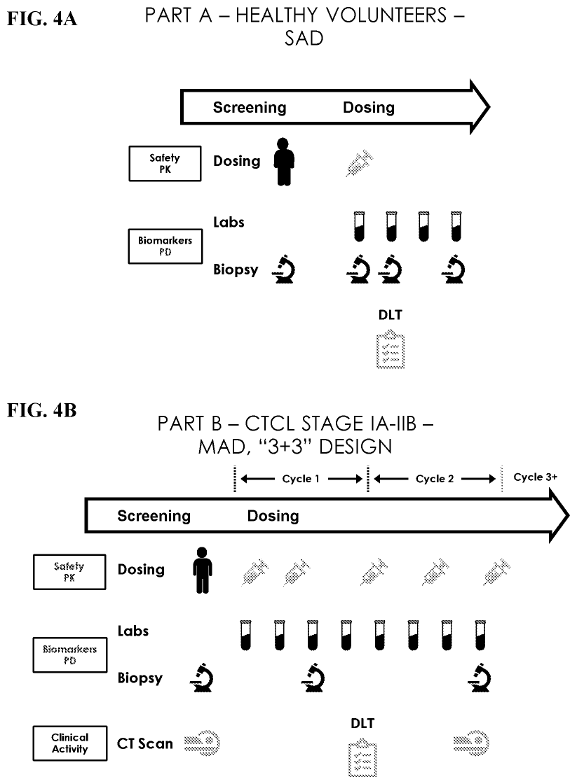

A clinical study in ongoing to test the safety and efficacy of treating a cancer in a human subject by administering engineered exosomes comprising surface displayed IL-12 (FIGS. 4A-4B). In part A, healthy volunteers will be administered varying doses of exoIL-12 and monitored of adverse events and biomarkers (FIG. 9A). Patients will be administered exoIL-12 at dose of 0.3 μg, 1.0 μg, 3.0 μg, 6.0 μg, and 12.0 μg of the IL-12 polypeptide. In part B, subjects diagnosed with CTCL (stage IA-IIB) will be administered various doses of exoIL-12 and monitored for safety and biomarkers (FIG. 4B). Clinical activity will be monitored by one or more CT scan. Subjects having CTCL, TNBC, melanoma, GBM, MCC, and/or Kaposi sarcoma will be eligible for part B of the trial.

In the CTCL portion of the study, two patients have been treated, each of whom has received twenty doses of exoIL-12 (6.0 g of the IL-12 polypeptide) across multiple lesions. Duration of treatment has been greater than six months, and no treatment emergent adverse events Grade 3 or higher or SAEs were observed and no dose modifications were required. Notably, no chills, fever, fatigue, dizziness, myalgia, headache or back pain were reported. These symptoms have been observed in previous clinical studies of subcutaneously administered rIL-12 at comparable doses (ranging from 2 to 12 g) to those used in the present study of exoIL-12].

exoIL-12 demonstrated improvement in overall disease, with regression ranging from 20-60% in all injected and non-injected lesions, resulting in a partial response in Patient 001 according to the CAILs score which is a registrational endpoint. Patient 001 received 17+ injections across 3 lesions: all lesions resolved and additional injections were deemed unnecessary by the treating physician. Patient 002 received 16+ injections across 2 lesions and is on study and continuing to receive injections.

Plasma pharmacokinetic (PK) measurements of both healthy volunteers and patients that received exoIL-12 showed no systemic exposure with levels of IL-12 below the limit of quantification. In contrast, previous rIL-12 clinical studies showed dose-dependent systemic exposure with dosages of 5 and 12 g resulting in Cₘₐₓ plasma levels of approximately 15 to 45 μg/ml within 6 to 12 hours after dosing.

### Example 3: Measurement of IL-12

Payload with Exosomes (IL-12 is Endogenously Loaded by the HEK293 Producer Cell Line)

### Free and Total IL-12 by AlphaLISA

The AlphaLISA (“Amplified luminescent proximity homogenous assay” linked immunosorbent assay) technique is commonly used to measure protein-protein interactions or the presence of a protein analyte in a homogenous, no-wash format. In this assay, an anti-IL-12 antibody conjugated acceptor bead is mixed with a solution of biotinylated anti-IL-12 antibody and streptavidin-coated donor beads. The presence of IL-12 in CB-102 drug substance samples causes the beads to be brought into proximity through interaction of each of the antibodies with IL-12. Excitation of the donor beads at 680 nm causes transfer of singlet oxygen to the acceptor beads and subsequent fluorescence at 615 nm. The magnitude of Alpha Signal at 615 nm is directly related to the concentration of IL-12 in the sample being detected.

The sample signal is measured relative to a standard curve of recombinant human IL-12 to determine IL-12 content in CB-102 samples. CB-102 drug substance samples are diluted to 5×10¹⁰ particles/mL and either measured directly for total IL-12 content or are centrifuged through a 96-well Agilent 300 kDa filter plate for measurement of free IL-12 content in the flowthrough.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

The present disclosure provides, inter alia, compositions of exosomes encapsulating STING agonists for use as therapeutics. The present disclosure also provides methods of producing exosomes encapsulating STING agonists and methods of administering such exosomes as therapeutics. While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed:

1. A method of preventing or treating a tumor in a subject in need thereof comprising administering to the subject a dose of an IL-12 polypeptide, wherein the amount of the IL-12 polypeptide in the dose is about 0.1 μg to about 30 μg.

2. The method of claim 1, wherein the IL-12 polypeptide is delivered by an extracellular vesicle.

3. The method of claim 1 or 2, wherein the IL-12 polypeptide is associated with an extracellular vesicle.

4. The method of any one of claims 1 to 3, wherein the dose comprises at least about 0.1 μg, at least about 0.2 μg, at least about 0.3 μg, at least about 0.4 μg, at least about 0.5 μg, at least about 0.6 μg, at least about 0.7 μg, at least about 0.8 μg, at least about 0.9 μg, at least about 1.0 μg, at least about 1.1 μg, at least about 1.2 μg, at least about 1.3 μg, at least about 1.4 μg, at least about 1.5 μg, at least about 1.6 μg, at least about 1.7 μg, at least about 1.8 μg, at least about 1.9 μg, at least about 2.0 μg, at least about 2.1 μg, at least about 2.2 μg, at least about 2.3 μg, at least about 2.4 μg, at least about 2.5 μg, at least about 2.6 μg, at least about 2.7 μg, at least about 2.8 μg, at least about 2.9 μg, at least about 3.0 μg, at least about 3.1 μg, at least about 3.2 μg, at least about 3.3 μg, at least about 3.4 μg, at least about 3.5 μg, at least about 3.6 μg, at least about 3.7 μg, at least about 3.8 μg, at least about 3.9 μg, at least about 4.0 μg, at least about 4.1 μg, at least about 4.2 μg, at least about 4.3 μg, at least about 4.4 μg, at least about 4.5 μg, at least about 4.6 μg, at least about 4.7 μg, at least about 4.8 μg, at least about 4.9 μg, at least about 5.0 μg, at least about 5.1 μg, at least about 5.2 μg, at least about 5.3 μg, at least about 5.4 μg, at least about 5.5 μg, at least about 5.6 μg, at least about 5.7 μg, at least about 5.8 μg, at least about 5.9 μg, at least about 6.0 μg, at least about 6.5 μg, at least about 7.0 μg, at least about 7.5 μg, at least about 8.0 μg, at least about 8.5 μg, at least about 9.0 μg, at least about 9.5 μg, at least about 10.0 μg, at least about 10.5 μg, at least about 11.0 μg, at least about 11.5 μg, at least about 12.0 μg, at least about 12.5 μg, at least about 13.0 μg, at least about 14 μg, at least about 15 μg, at least about 16 μg, at least about 17 μg, at least about 18 μg, at least about 19 μg, at least about 20 μg, at least about 25 μg, or at least about 30 μg of the IL-12 polypeptide.

5. The method of any one of claims 1 to 3, wherein the dose comprises at least about 0.3 μg of the IL-12 polypeptide.

6. The method of any one of claims 1 to 3, wherein the dose comprises at least about 1.0 μg of the IL-12 polypeptide.

7. The method of any one of claims 1 to 3, wherein the dose comprises at least about 3.0 μg of the IL-12 polypeptide.

8. The method of any one of claims 1 to 3, wherein the dose comprises at least about 6.0 μg of the IL-12 polypeptide.

9. The method of any one of claims 1 to 3, wherein the dose comprises at least about 12.0 μg of the IL-12 polypeptide.

10. The method of any one of claims 1 to 9, wherein the dose of the IL-12 polypeptide is administered at least two times, at least three times, at least four times, at least five times, or at least six times.

11. The method of any one of claims 1 to 10, wherein the dose of the IL-12 polypeptide is administered once about every week, once about every two weeks, once about every three weeks, once about every four weeks, once about every six weeks, or once about every eight weeks.

12. The method of any one of claims 1 to 11, wherein the IL-12 polypeptide comprises a single chain polypeptide having at least about 70%, at least about 75%, at least about 80%, at least about 85%, at least about 90%, at least about 95%, at least about 96%, at least about 97%, at least about 98%, at least about 99%, or about 100% sequence identity to SEQ ID NO: 13.

13. The method of any one of claims 1 to 12, wherein the extracellular vesicle comprises a scaffold moiety.

14. The method of claim 13, wherein the scaffold moiety comprises a Prostaglandin F2 receptor negative regulator (PTGFRN) protein or a portion thereof.

15. The method of claim 14, wherein the PTGFRN protein comprises SEQ ID NO: 33.

16. The method of claim 14, wherein the PTGFRN protein comprises at least about 70%, at least about 85%, at least about 90%, at least about 95%, at least about 96%, at least about 97%, at least about 98%, at least about 99%, or about 100% identical to SEQ ID NO: 1.

17. The method of claim 16, wherein the PTGFRN protein comprises the amino acid sequence as set forth in SEQ ID NO: 1.

18. The method of any one of claims 13 to 17, wherein the IL-12 polypeptide is linked to the scaffold moiety.

19. The method of claim 18, wherein the IL-12 polypeptide is linked to the scaffold moiety by a peptide bond.

20. The method of any one of claims 1 to 19, wherein the IL-12 is associated with the exterior surface of the extracellular vesicle.

21. The method of any one of claims 13 to 20, wherein the IL-12 polypeptide is linked to the N-terminus of the scaffold moiety.

22. The method of any one of claims 13 to 21, wherein the IL-12 polypeptide is linked to the N-terminus of the PTGFRN protein of the portion thereof.

23. The method of any one of claims 1 to 22, wherein the IL-12 is within the lumen of the extracellular vesicle.

24. The method of any one of claims 13 to 24, wherein the IL-12 polypeptide is linked to the C-terminus of the scaffold moiety.

25. The method of any one of claims 13 to 24, wherein the IL-12 polypeptide is linked to the C-terminus of the PTGFRN protein of the portion thereof.

26. The method of any one of claims 1 to 25, wherein the amount of IL-12 polypeptide is measured by fluorescence assay, IL-12 AlphaLISA, or a combination thereof.

27. The method of any one of claims 1 to 26, wherein the tumor is a primary tumor, a secondary tumor, or both a primary tumor and a secondary tumor.

28. The method of any one of claims 1 to 27, wherein the administering reduces the volume of the tumor.

29. The method of any one of claims 1 to 28, wherein the administering reduces the volume of the tumor by at least two fold, at least three fold, at least four fold, at least five fold, at least six fold, at least seven fold, at least nine fold, or at least ten fold compared to the tumor volume after administering the IL-12 polypeptide in the absence of an extracellular vesicle.

30. The method of claim 28 or 29, wherein the administering reduces the volume of the primary tumor.

31. The method of claim 30, wherein the administering is capable of reducing the volume of the primary tumor by at least about 1.5 fold, at least about 2 fold, at least about 3 fold, at least about 4 fold, or at least about 5 fold compared to the monotherapy after day 14 of the administering.

32. The method of any one of claims 1 to 31, wherein the administering reduces the growth of the tumor.

33. The method of any one of claims 1 to 32, wherein the administering reduces the growth of the tumor by at least two fold, at least three fold, at least four fold, at least five fold, at least six fold, at least seven fold, at least nine fold, or at least ten fold compared to the tumor volume after administering either an extracellular vesicle comprising the STING agonist or the IL-12 polypeptide (“monotherapy”).

34. The method of any one of claims 1 to 33, further comprising administering an additional anti-cancer agent.

35. The method of claim 34, wherein the additional anti-cancer agent comprises a checkpoint inhibitor.

36. The method of claim 35, wherein the checkpoint inhibitor comprises an anti-PD-1 antibody, an anti-PD-L1 antibody, an anti-CTLA-4 antibody, an anti-LAG-3 antibody, an anti-TIM-3 antibody, or any combination thereof.

37. The method of claim 36, wherein the checkpoint inhibitor is an anti-PD-1 antibody.

38. The method of any one of claims 2 to 37, wherein the extracellular vesicle is an exosome, a nanovesicle, an apoptotic body, a microvesicle, a lysosome, an endosome, a liposome, a lipid nanoparticle, a micelle, a multilamellar structure, a revesiculated vesicle, or an extruded cell.

39. The method of any one of claims 2 to 38, wherein the EV is an exosome.

40. The method of any one of claims 2 to 39, wherein the extracellular vesicle is produced by a cell that overexpresses a PTGFRN protein.

41. The method of any one of claims 2 to 40, wherein the extracellular vesicle further comprises a ligand, a cytokine, or an antibody.

42. The method of claim 41, wherein the antibody comprises an antagonistic antibody and/or an agonistic antibody.


SEQUENCE LISTING (403 sequences)

SEQ ID NO: 1 - protein, 879 aa, Homo sapiens
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2 - protein, 731 aa, synthetic construct
  REGION 1..731: note = Synthetic
PSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 3 - protein, 611 aa, synthetic construct
  REGION 1..611: note = Synthetic
VATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 4 - protein, 485 aa, synthetic construct
  REGION 1..485: note = Synthetic
SPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 5 - protein, 343 aa, synthetic construct
  REGION 1..343: note = Synthetic
KPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 6 - protein, 217 aa, synthetic construct
  REGION 1..217: note = Synthetic
VRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 7 - protein, 66 aa, synthetic construct
  REGION 1..66: note = Synthetic
SKPVFITVKMDVLNAFKYPLLIGVGLSTVIGLLSCLIGYCSSHWCCKKEVQETRRERRRLMSMEMD

SEQ ID NO: 8 - protein, 21 aa, synthetic construct
  REGION 1..21: note = Synthetic
MGRLASRPLLLALLSLALCRG

SEQ ID NO: 9-10 - skipped (2 entries)

SEQ ID NO: 11 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 12 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 13 - protein, 548 aa, synthetic construct
  REGION 1..548: note = Synthetic
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGGSGGGGSGGGGSGGGSGGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 14-32 - skipped (19 entries)

SEQ ID NO: 33 - protein, 192 aa, Homo sapiens
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-400 - skipped (367 entries)

SEQ ID NO: 401 - protein, 332 aa, synthetic construct
  REGION 1..332: note = Synthetic
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 402 - protein, 195 aa, synthetic construct
  REGION 1..195: note = Synthetic
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 403 - protein, 227 aa, synthetic construct
  REGION 1..227: note = Synthetic
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE